(19) United States
(12) Patent Application Publication
(10) Pub. No.: US 2026/0077037 A1
(43) Pub. Date: Mar. 19, 2026

(54) CATIONIC LIPID BASED COMPOSITION, FORMULATION AND USE FOR NUCLEIC ACID VACCINE DELIVERY AND PREPARATION THEREOF

(71) Applicants: BHARAT BIOTECH INTERNATIONAL LIMITED, Hyderabad (IN); COUNCIL OF SCIENTIFIC AND INDUSTRIAL RESEARCH, New Delhi (IN)

(72) Inventors: Namita Santosh MAHADIK, Telangana (IN); Bhattacharyya TITHI, Telangana (IN); Bhowmira RATHORE, Telangana (IN); Pooja POTDAR, Telangana (IN); Immanuel CHRISTIANA, Telangana (IN); Sabarinadh CHILAKA, Telangana (IN); Sai Balaji ANDUGULAPATI, Telangana (IN); Ramakrishna SISTLA, Telangana (IN); Rajkumar BANERJEE, Telangana (IN); Krishna Murthy ELLA, Telangana (IN); D. Yogeswara RAO, Telangana (IN)

(73) Assignees: BHARAT BIOTECH INTERNATIONAL LIMITED, Hyderabad (IN); COUNCIL OF SCIENTIFIC AND INDUSTRIAL RESEARCH, New Delhi (IN)

(21) Appl. No.: 18/881,055

(22) PCT Filed: Jun. 22, 2023
(86) PCT No.: PCT/IN2023/050596

§ 371 Date: Jan. 3, 2025

(30) Foreign Application Priority Data
Jul. 4, 2022 (IN) 202241038333

(51) Int. Cl. A61K 39/385; A61K 39/00; A61K 39/12; A61K 39/125; A61K 39/145; A61K 39/15; A61K 39/155; A61K 39/205; A61K 39/21; A61K 39/215; A61K 39/235; A61P 37/04

(52) U.S. Cl. CPC A61K 39/385; A61K 39/12; A61K 39/125; A61K 39/145; A61K 39/15; A61K 39/155; A61K 39/205; A61K 39/21; A61K 39/215; A61K 39/235; A61P 37/04; A61K 2039/53; A61K 2039/6018

(57) ABSTRACT

The present invention discloses a cationic lipid-based formulation for in vivo delivery of nucleic acid. The present invention describes the development of a lipid system that can induce efficient non-viral delivery of nucleic acid. especially RNA. for the purpose of efficient nucleic acid transfection toward eliciting vaccination in vivo. Present invention provides a lipid/RNA complex (lipoplex) formulation having sufficiently long shelf life that performs without any compromise in its transfection output. The present invention further provides cationic lipid-based formulations for RNA delivery with maximum nucleic acid complexation ability and with comparatively lesser amount of cationic lipid having higher stability.

### CROSS-REFERENCE TO RELATED APPLICATION(S)

This application claims the priority to, and benefit of Indian Complete patent application No. 202241038333 filed on Jul. 4, 2022: the disclosures of which are incorporated herein by reference.

### SEQUENCE LISTING

A sequence listing in electronic (XML file) format is filed with this application and incorporated herein by reference. The name of the XML file is “Sequence_listing_2005A.xml”: the file was created on Jan. 2, 2025: the size of the file is 2,748 bytes.

### FIELD OF THE INVENTION

The present invention relates to lipid composition for delivery of therapeutic agents. Particularly, the present invention relates to cationic lipid-based composition for in vivo delivery of nucleic acid. More particularly, the present invention relates to cationic lipid-based composition, formulation and use for nucleic acid vaccine delivery and preparation thereof. The present invention describes the development of a lipid system that can induce efficient non-viral delivery of nucleic acid, especially RNA, for the purpose of efficient nucleic acid transfection toward eliciting vaccination in vivo.

### BACKGROUND OF THE INVENTION

Health problems and mortality associated with virus infections are of tremendous concern internationally. Currently, messenger ribonucleic acid (mRNA)-based drugs, remarkably mRNA vaccines have been widely proven to be one of the most promising treatment strategies in immune therapeutics. Therefore, there has been increasing focus on the use of messenger ribonucleic acid (mRNA) drugs or mRNA vaccines as a new therapeutic modality. Current clinical efforts encompassing mRNA-based drugs are directed toward infectious disease vaccines, cancer immunotherapies, therapeutic protein replacement therapies, and treatment of genetic diseases (Opportunities and Challenges in the Delivery of mRNA-Based Vaccines, Pharmaceutics: 2020 February: 12 (2): 102).

The mRNA vaccines contain a single-stranded RNA molecule that complements DNA. The broad advantages associated with the mRNA vaccines are their high efficacy, relatively low severity of side effects, and low attainment costs. These properties have enabled mRNA vaccines to become emergent therapeutics as evident from the databases on pre-clinical and clinical trials against various infectious diseases, cancers, and as potential gene therapeutic tools. The genetic signature implanted in an artificial or synthetic mRNA would confer ability to tailor-make the alteration for its potential uses against many diseases with minimal modifications. Overall, flexible design, standardized production processes, and relatively short-lived cytoplasmic presence make mRNA vaccines very powerful, especially in a pandemic situation with rapidly mutating viruses.

The strategy to leverage mRNA science focuses on creating a bench platform that can amalgamate the multidisciplinary technology to target infectious and non-infectious diseases.

In general when the therapeutic agents (such as nucleic acid) are delivered to subjects, there are many difficulties faced by the therapeutic agents reaching the target cell or tissue. Entry of these therapeutic agents into living cells is highly restricted by the complex membrane systems of the cells. In order to overcome these restrictions, higher concentrations of therapeutic agents more than the required amount to achieve the result is needed. But this increases the risk of toxic effects and side effects of these higher concentrations of therapeutic agents.

One solution to this problem is to utilize delivery systems that allows selective entry into the cell. To function in vivo, therapeutic agents (such as nucleic acid) require safe, effective, and stable delivery systems that protect the nucleic acid from degradation and that allow cellular uptake and release. These delivery systems include specific carrier molecules such as Lipid carriers, biodegradable polymers and various conjugate systems that can improve delivery of therapeutic agents to target cell or tissue. The therapeutics is entirely dependent on delivery systems which comprises lipids, polymers etc. Lipids that find maximum use for such deliveries are cationic lipids in their original form or are ionizable to form cationic lipids at low pH. The lipids spontaneously form aggregates and carry surface positive charges that make electrostatic complexation with nucleic acids, which are negatively charged. As the lipids are synthesized employing robust organic chemical synthesis, large scale production of such molecules and hence up-scaling of lipid/nucleic acid complexation product for vaccination are technologically and commercially very much feasible.

Among the existing vaccination protocols, lipid-based nucleic acid (DNA, m-RNA) delivery systems hold high promise and versatility. Recently, among nucleic acid therapeutics, RNA therapeutics is gaining attention and has shown great potential in treating diseases which are being fundamentally different than using small molecules and conventional immunological approaches.

However, one of the greatest challenges encountered when developing the lipid/RNA complex (lipoplex) is their poor stability. The lipoplex formed from commercial formulations need sub-zero temperature for preservation toward maintaining its activity. It is difficult to widely distribute and store the existing lipoplex and such mRNA vaccines and formulations at ultra-low temperatures (e.g., −20° C. to −80° C.) above which the lipoplex becomes unstable and becomes impractical for long time storage. Hence, it needs more expensive storage conditions, thus demanding higher shipment and storage cost. In order to effectively distribute a vaccine widely, the mRNA vaccines should have a sufficiently long shelf life without any compromise in its transfection output, thus enabling a situation tenable for long-time, low-cost storage, and transportation.

In addition, the existing cationic-ionizable lipid-based non-viral formulations generally work at higher N/P ratio i.e., lipid to RNA ratio. The existing commercial formulations use N/P charge ratio of ≥6:1. This ratio varies from 6:1 to 10:1. Cationic lipid although not tested but may have some toxicity. Thus, more lipid nanoparticles (LNPs) are required, which can cause unwanted side effects such as pain, swelling, fever, and sleepiness in vivo. Sonia Ndeupen et. al., “The mRNA-LNP platform's lipid nanoparticle component used in preclinical vaccine studies is highly inflammatory”, iScience, Volume 24, Issue 12, highlights such side effects towards use the high LNPs that are responsible for these side effects. In order to effectively reduce the side effects, the lipid/RNA complex (lipoplex) should have an optimized N/P ratio, as low as possible in order to reduce side effects in vivo. Further to reduce the lipid-mediated toxicity, its longer body exposure shall also be minimized.

Therefore, keeping in view the hitherto prior art, there is a need for a lipid delivery system that can induce efficient non-viral delivery of nucleic acid, especially RNA, for the purpose of efficient nucleic acid transfection towards eliciting vaccination in vivo.

### OBJECTS OF THE INVENTION

Primary object of the invention is to provide a cationic lipid (CL) based composition, formulation and use for nucleic acid vaccine delivery and preparation thereof.

Another object of the invention is to provide a lipid system to induce efficient non-viral delivery of nucleic acid, especially RNA, for the purpose of efficient nucleic acid transfection toward eliciting vaccination in vivo.

Another object of the invention is to provide a cationic lipid (CL) based composition formulated in a suitable formulation which is used to deliver a biologically active agent as a candidate vaccine antigen in combination with one or more other lipid components and optionally one or more excipients.

Another object of the invention is to provide safe, effective, and stable delivery systems that protect the nucleic acid from degradation that allow cellular uptake and release.

Another object of the invention is to provide highly stable cationic lipid (CL) based composition at room temperature.

Another object of the invention is to provide a lipid/RNA complex (lipoplex) having sufficiently long shelf life that performs without any compromise in its transfection output.

Another object of the invention is to provide a cationic lipid-based composition having low-cost storage, and transportation cost.

Another object of the invention is to provide lipid-based composition for nucleic acid delivery system that enables efficient in vivo transfection of nucleic acid with low N/P ratio.

Another object of the invention is to provide a lipid-based composition and/or formulation that minimizes lipid-mediated toxicity in a subject administered with, by minimizing the amount of cationic-lipid required in the composition/formulation.

Yet another object of the invention is to provide a vaccine formulation for the prophylaxis and/or treatment of virus-mediated disease in a human subject.

A further object of the invention is to provide cationic lipid-based compositions for RNA delivery with maximum nucleic acid complexation ability and with comparatively lesser amount of cationic lipid having higher stability.

### SUMMARY OF THE INVENTION

The present invention relates to cationic lipid-based composition, formulation and use for nucleic acid vaccine delivery and preparation thereof.

Accordingly, the present invention discloses and describes the development of a lipid system that can induce efficient non-viral delivery of nucleic acid for the purpose of efficient nucleic acid transfection toward eliciting vaccination in vivo. The present invention discloses and provides a cationic lipid (CL) based composition comprising at least one cationic lipid of N, N di-octadecyl, N, N dihydroxyethyl ammonium chloride [lipid D0] or N-oleyl, N-octadecyl, N, N dihydroxyethyl ammonium chloride [lipid D1], that is formulated in a suitable formulation which is used to deliver a biologically active agent as a candidate vaccine antigen such as nucleic acid derived vaccine antigen, in combination with one or more other lipid components and optionally one or more excipients.

In one aspect the present invention provides novel cationic lipid (CL) based composition for nucleic acid (NA) delivery, wherein the CL-based composition comprises:
(a) Cationic lipid: N-oleyl, N-octadecyl, N, N dihydroxyethyl ammonium chloride [lipid D1] or its salt, solvate, derivatives, or combination thereof:
(b) combination of Neutral/Zwitter-ionic co-lipids which comprises two or more lipids selected from:
   (b1) steroid lipid which may be sterol and is selected from Cholesterol, ergosterol, stigmasterol, sitosterol, campesterol, stigmastanol like phytosterols and other steroids such as dexamethasone, prednisolone, triamcinolone and/or mixture thereof:
   (b2) phospholipid which may be selected from Dioleoyl phosphatidylethanolamine [DOPE], 1,2-Dioleoyl-sn-Glycero-3-Phosphocholine [DOPC], 1,2-Distearoyl-sn-glycero-3-phosphoethanolamine [DSPE], 1,2-Distearoyl-sn-glycero-3-phosphocholine [DSPC], 1,2-Dimyristoyl-sn-glycero-3-phosphoethanolamine [DMPE], 1,2-Dimyristoyl-sn-glycero-3-phosphocholine [DMPC], 1,2-Dipalmitoyl-sn-glycero-3-phosphoethanolamine [DPPE], 1,2-Dipalmitoyl-sn-glycero-3-phosphocholone [DPPC] or mixture thereof, preferably DOPE and/or DOPC; and (b3) a phospholipid-polymer conjugate which may be selected from conjugates prepared by conjugation of a suitable phospholipid selected from Dioleoyl phosphatidylethanolamine [DOPE], 1,2-Distearoyl-sn-glycero-3-phosphoethanolamin [DSPE], 1,2-Dimyristoyl-sn-glycero-3-phosphoethanolamine [DMPE], 1,2-Dipalmitoyl-sn-glycero-3-phosphoethanolamin [DPPE], with a suitable polymer which is selected from polyethylene-glycol [PEG] of 2000, 5000, 8000 Average molecular weight, and
(c) optionally, one or more pharmaceutically acceptable excipient.

In one embodiment, in the said cationic lipid (CL) based composition for nucleic acid (NA) delivery of the present invention the steroid lipid (b1) is cholesterol.

In one embodiment, in the said cationic lipid (CL) based composition for nucleic acid (NA) delivery of the present invention the phospholipid (b2) is Dioleoyl phosphatidylethanolamine [DOPE].

In one embodiment, in the said cationic lipid (CL) based composition for nucleic acid (NA) delivery of the present invention the Phospholipid-Polymer conjugate (b3) is 1,2-distearoyl-sn-glycero-3-phosphoethanolamine-N-[amino(polyethylene glycol)-2000 [DSPE-PEG].

In one embodiment, the cationic lipid (CL) based composition for nucleic acid (NA) delivery of the present invention comprises:
(a) cationic lipid N-oleyl, N-octadecyl, N, N dihydroxyethyl ammonium chloride [lipid-D1]:
(b) neutral/zwitter-ionic co-lipids selected from (b1) Cholesterol, (b2) Dioleoylphosphatidylethanolamine [DOPE], with or without Phospholipid-Polymer conjugate (b3) 1,2-distearoyl-sn-glycero-3-phosphoethanolamine-N-[amino(polyethylene glycol)-2000 [DSPE-PEG]; and
(c) optionally, one or more pharmaceutically acceptable excipient.

In one embodiment, in the said cationic lipid (CL) based composition for nucleic acid (NA) delivery of the present invention the mole ratio of N-oleyl, N-octadecyl, N, N dihydroxyethyl ammonium chloride [Lipid-D1], Cholesterol, 1,2-Dioleoyl-sn-glycero-3-phosphoethanolamine [DOPE] in the composition is 1:1:1.

In one embodiment, in the said cationic lipid (CL) based composition for nucleic acid (NA) delivery of the present invention the mole ratio of N-oleyl, N-octadecyl, N, N dihydroxyethyl ammonium chloride [Lipid-D1], Cholesterol, 1,2-Dioleoyl-sn-glycero-3-phosphoethanolamine [DOPE], and 1,2-distearoyl-sn-glycero-3-phosphoethanolamine-N-[amino(polyethylene glycol)-2000 [DSPE-PEG] in the composition is 1:1:1:0.03.

The Nucleic Acid (NA) in the said cationic lipid (CL) based composition, is a biologically active agent which is delivered to a subject in need thereof using the said CL composition, wherein the Nucleic Acid (NA) is selected from ribonucleic acid (RNA), messenger RNA (mRNA), Deoxyribonucleic acid (DNA), plasmid DNA (pDNA), fragment of RNA, mRNA, DNA, pDNA, miRNA or any chimeric or fusion thereof.

The said Nucleic Acid (NA) is derived from a living bio-organism and non-living bio-organism.

In one embodiment, the biologically active agent is Nucleic Acid (NA) derived from a virus which may be selected from Coronaviridae, Retroviridae, Reoviridae, Togaviridae, Filoviridae, Flavirideae, Papillomaviridae, Plasmodiidae, Hepadnaviridae, Picornaviridae, Caliciviridae, Orthomyxoviridae, Adenoviridae, Rhabdoviridae, Paramyxoviridae, Orthomyxoviridae, and Nodaviridae.

In one embodiment, the biologically active agent is a DNA, pDNA, RNA, mRNA derived from the virus selected from Coronaviridae, Retroviridae, Reoviridae,

Togaviridae, Filoviridae, Flavirideae, Papillomaviridae, Plasmodiidae, Hepadnaviridae, Picornaviridae, Caliciviridae, Orthomyxoviridae, Adenoviridae, Rhabdoviridae, Paramyxoviridae, Orthomyxoviridae and Nodaviridae

In one embodiment, the cationic lipid (CL) based composition of the present invention, the composition for biologically active Nucleic Acid (NA) delivery is formulated in a suitable lipid formulation preparation as a delivery system with or without one or more pharmaceutically acceptable excipient, wherein the suitable lipid formulation may be selected from:
Nanoparticle formulation,
Lipid/Liposome/Liposomal formulation,
Lipoplex formulation,
Lipid/polymer emulsion formulation.

The composition of the present invention is formulated as a cationic lipid (CL) based formulation formulated along with the biologically active Nucleic Acid (NA) to provide a pharmaceutical formulation with or without one or more pharmaceutically acceptable excipient, to administer in a subject in need thereof, wherein the pharmaceutical formulation comprises:
a Lipid-Nanoparticle (LNP) formulation or a Liposomal formulation—encapsulating the biologically active agent, or
a Lipoplex formulation—formed by complexion of Liposomal preparation with the biologically active agent.

The pharmaceutically acceptable excipients in the said composition are selected from buffers, adjuvants, diluents, lubricants, binders, stabilizers, preservatives, disintegrants, absorbents, colorants surfactants, flavors, sweeteners. residuals, immune boosting co-excipients or combination thereof.

The pharmaceutical formulation of the present invention is a vaccine formulation for delivery of biologically active agent which comprises:
a complex of Lipid composition+DNA/pDNA (DNA/pDNA-Lipoplex), or
a complex of Lipid composition+RNA/mRNA complex (RNA/mRNA-Lipoplex).

The said formulation of the present invention performs in vivo transfection at a N/P charge ratio (Lipid:Nucleic acid) of 1:1 to 4:1.

Preferably the N/P charge ratio is 1:1 or 2:1 or 3:1 or 4:1.

The present formulation is a lipid/mRNA complex (lipoplex) which is stable at room temperature for at least 30 days.

The cationic lipid (CL) based composition, or the cationic lipid (CL) based formulation including the vaccine formulation of the present invention, minimizes lipid-mediated toxicity in a subject administered with, by minimizing the amount of cationic-lipid required in the composition/formulation.

In another aspect present invention discloses a vaccine comprising the cationic lipid (CL) based composition or the cationic lipid (CL) based formulation for the prophylaxis and/or treatment of virus mediated disease in a human subject.

Accordingly present invention discloses a vaccine formulation comprising nucleic acid as vaccine antigen for the prophylaxis and/or treatment of virus-mediated disease in a human subject, wherein the vaccine comprises:
a Nucleic acid (NA) based biologically active vaccine antigen selected from DNA, pDNA, RNA, mRNA, fragment, or combination thereof derived from virus:
a cationic-based (CL)-lipid composition which comprises
   a. Cationic lipid: N-oleyl, N-octadecyl, N, N dihydroxyethyl ammonium chloride [lipid D1] or its salt, solvate, derivatives, or combination thereof; and
   b. combination of Neutral/Zwitter-ionic co-lipids which comprises two or more lipids selected from:
      (b1) steroid lipid which may be sterol and is selected from Cholesterol, ergosterol, stigmasterol, sitosterol, campesterol, stigmastanol like phytosterols and other steroids such as dexamethasone, prednisolone, triamcinolone and/or mixture thereof:
      (b2) phospholipid which may be selected from Dioleoyl [DOPE], 1,2-Dioleoyl-sn-Glycero-3-phosphatidylethanolamine Phosphocholine [DOPC], 1,2-Distearoyl-sn-glycero-3-phosphoethanolamine [DSPE], 1,2-Distearoyl-sn-glycero-3-phosphocholine [DSPC], 1,2-Dimyristoyl-sn-glycero-3-phosphoethanolamine [DMPE], 1,2-Dimyristoyl-sn-glycero-3-phosphocholine [DMPC], 1,2-Dipalmitoyl-sn-glycero-3-phosphoethanolamine [DPPE], 1,2-Dipalmitoyl-sn-glycero-3-phosphocholone [DPPC] or mixture thereof, preferably DOPE and/or DOPC; and
      (b3) a phospholipid-polymer conjugate which may be selected from conjugates prepared by conjugation of a suitable phospholipid selected from Dioleoyl phosphatidylethanolamine [DOPE], 1,2-Distearoyl-sn-glycero-3-phosphoethanolamin [DSPE], 1,2-Dimyristoyl-sn-glycero-3-phosphoethanolamine [DMPE], 1,2-Dipalmitoyl-sn-glycero-3-phosphoethanolamin [DPPE], with a suitable polymer which is selected from polyethylene-glycol [PEG] of 2000, 5000, 8000 Average molecular weight, and
   c. optionally, one or more pharmaceutically acceptable excipient.

In the said vaccine formulation, the vaccine antigen DNA, pDNA, RNA, mRNA, fragment, or combination thereof are derived from virus selected from Coronaviridae, Retroviridae, Reoviridae, Togaviridae, Filoviridae, Flavirideae, Papillomaviridae, Plasmodiidae, Hepadnaviridae, Picornaviridae, Caliciviridae, Orthomyxoviridae, Adenoviridae, Rhabdoviridae, Paramyxoviridae, Orthomyxoviridae or Nodaviridae.

The vaccine formulation of the present invention wherein the vaccine antigen is mRNA formulated with a suitable lipid formulation for delivery of active antigenic agent which comprises:
(a) cationic lipid N-oleyl, N-octadecyl, N, N dihydroxyethyl ammonium chloride [lipid-D1]:
(b) neutral/zwitter-ionic co-lipids selected from (b1) Cholesterol, (b2) Dioleoylphosphatidylethanolamine [DOPE], with or without Phospholipid-Polymer conjugate (b3) 1,2-distearoyl-sn-glycero-3-phosphoethanolamine-N-[amino(polyethylene glycol)-2000 [DSPE-PEG]; and
(c) optionally, one or more pharmaceutically acceptable excipient.

In the said vaccine formulation the mole ratio of N-oleyl, N-octadecyl, N, N dihydroxyethyl ammonium chloride [Lipid-D1], Cholesterol, 1,2-Dioleoyl-sn-glycero-3-phosphoethanolamine [DOPE] in the composition of the vaccine formulation is 1:1:1. In the said vaccine formulation the mole ratio of N-oleyl, N-octadecyl, N, N dihydroxyethyl ammonium chloride [Lipid-D1], Cholesterol, 1,2-Dioleoyl-sn-glycero-3-phosphoethanolamine [DOPE], and 1,2-distearoyl-sn-glycero-3-phosphoethanolamine-N-[amino(polyethylene glycol)-2000 [DSPE-PEG] in the composition of the vaccine formulation is 1:1:1:0.03.

The said vaccine formulation may be formulated with or without one or more pharmaceutically acceptable excipient.

The pharmaceutically acceptable excipients are selected from buffers, adjuvants, diluents, lubricants, binders, stabilizers, preservatives, disintegrants, absorbents, colorants surfactants, flavors, sweeteners. residuals, immune boosting co-excipients or combination thereof.

In the said vaccine composition, the suitable lipid formulation may be selected from:
Nanoparticle formulation,
Lipid/Liposome/Liposomal formulation,
Lipoplex formulation,
Lipid/polymer emulsion formulation.

In one embodiment the cationic lipid (CL) based formulation in the said vaccine formulation of the present invention forms a Liposomal formulation.

In another embodiment the cationic lipid (CL) based formulation in the said vaccine formulation of the present invention forms a Lipid nanoparticle formulation.

In yet another embodiment the cationic lipid (CL) based formulation in the said vaccine formulation of the present invention forms a Lipoplex.

The vaccine formulation of the present invention, wherein the suitable lipid formulation is selected from:
a Lipid-Nanoparticle (LNP) formulation or a Liposomal formulation—encapsulating the mRNA,
a Lipoplex formulation—formed by complexion of Liposomal preparation with mRNA.

The said vaccine formulation performs in vivo transfection at a N/P charge ratio (Lipid:mRNA) of 1:1 to 4:1. Preferably, the N/P charge ratio is 1:1 or 2:1 or 3:1 or 4:1.

The vaccine formulation of the present invention is stable at room temperature for at least 30 days.

The vaccine formulation of the present invention minimizes lipid-mediated toxicity in a subject administered with vaccine in need thereof, by minimizing the amount of cationic-lipid required in the composition/formulation of the vaccine.

The vaccine formulation of the present invention is in the liquid, powder, lyophilized, suspension form.

In another aspect present invention discloses a method for preparation of a cationic lipid (CL) based composition, comprising the steps of:
a) taking CL and other co-lipids and excipients first in chloroform and mixing thoroughly in a vial;
b) drying off chloroform by thin layer of N₂ flow, followed by keeping the vial at high vacuum; and
c) dissolving the residual lipid mixture in minimal ethanol followed by rapidly injecting the ethanolic lipid mixture in the suitable buffer solution, typically of pH 7.4, to make the CL based formulation in room temperature.

In another aspect present invention discloses a method for preparation of vaccine formulation, comprising the steps of:
a) preparation of RNA construct by in vitro transcription process:
b) complexing the CL based formulation as describe above with RNA at a fixed mole ratio to make lipid/RNA complex or ‘lipoplex’:
c) obtaining the lipoplex as the vaccine solution and preserving it at room temperature or at 4° C.

In another aspect present invention discloses a method of prophylaxis, treatment, and management of virus-mediated disease by administering a vaccine formulation in a subject in the need thereof by intranasal, intraperitoneal, oral, intramuscular, subcutaneous or intradermal routes.

In yet another aspect present invention discloses use of cationic lipid (CL) based composition for:
delivery of Nucleic acid:
minimizing toxicity of cationic lipid:
in preparation of pharmaceutical formulation including vaccine formulation;
in prophylaxis, treatment, and management of diseases caused by living and non-living microorganism including bacteria and viruses.

The present invention is described in detail in below description, examples and represented in figures and as claimed in appended claims.

### BRIEF DESCRIPTION OF FIGURES

FIG. 1: Gel retardation assay to show level of lipid/nucleic acid (here, pDNA) complexation with varied charge (N/P) ratio

FIG. 2: m-RNA transfection using D0 and D1 based lipid formulations
A) With D0 lipid
B) With D1 lipid

FIG. 3: pDNA DNA transfection ability of D0 (CLF15, CLF17, CLF1) and D1 (CLF16, CLF18, CLF2)
A) With D0 lipid
B) With D1 lipid

FIG. 4:
A) mRNA synthesis
   1. N-Luc-mRNA (with ARCA)—0.4 μg+POLY (A)
   2. N-Luc-mRNA (with ARCA)—1 μg+POLY (A)
   3. N-Luc-mRNA (with ARCA)—without POLY (A)
B) Expression of N-Luc protein in mRNA transfected cells (N luc mRNA 1 sec)
C) Expression in in vivo (Day 1: N luc mRNA in Balb C mice)
D) Expression in in vivo (Day 3: N luc mRNA in Balb C mice)

FIG. 5: Expression of N-Luc protein in vivo on day 1 and 2 post injection

FIG. 6: In vivo study to depict the stability of N-luc mRNA while remain in complex with lipids CLF2 and CLF18 with respect to time at room temperature.
A) 30 min stability: left mice (none) CLF18; right mice left ear cut CLF 2
B) 24 hours stability: left mice (none) CLF18; right mice left ear cut CLF 2
C) 3^{rd} day stability: left mice (none) CLF18; right mice left ear cut CLF 2
D) 30^{th} day stability: Left side injected with CLF 18 formulation and right side injected with CLF-2 formulation

FIG. 7: In vivo study to depict the extent of residence for lipid over a period of time following IM injection
A) Day 1: Lipid without Dir Dye
B) Day 1: CLF2
C) Day 1: CLF18
D) Day 5: Lipid without Dir Dye
E) Day 5: CLF2
F) Day 5: CLF18
G) Day 15: CLF2
H) Day 15: CLF18

FIG. 8: Expression of luciferase protein
A) Imaged after 24 hours of injection
B) Imaged after 7 days of injection
C) Imaged after 30 days of injection

FIG. 9: Effect of RNAses on mRNA alone and mRNA complexed with CLF-2 borate buffer

FIG. 10: Nano-Luciferase Sequence (SEQ ID NO: 1) shown in Example 4.

### DETAILED DESCRIPTION OF THE INVENTION

The present invention focuses on creating a model platform for nucleic acid vaccines by designing, synthesis and delivery of various nucleic acid vaccines with high specificity and superior selectivity with negligible or no allergic reactions. The present invention discloses and describes the development of a lipid system that can induce efficient non-viral delivery of nucleic acid, especially RNA, for the purpose of efficient nucleic acid transfection toward eliciting vaccination in vivo.

The present invention is directed to a cationic lipid-based composition, formulation and use for nucleic acid vaccine delivery and preparation thereof.

In one aspect present invention discloses a cationic lipid (CL) based composition for nucleic acid (NA) delivery.

In one embodiment present invention discloses a cationic lipid (CL) based composition comprising cationic lipid N, N di-octadecyl, N, N dihydroxyethyl ammonium chloride [lipid D0] or N-oleyl, N-octadecyl, N, N dihydroxyethyl ammonium chloride [lipid D1] or their salt, solvate, derivatives, or combination thereof: along with various excipients mixed in different ratios.

In one embodiment present invention discloses a cationic lipid (CL) based composition comprising cationic lipid N, N di-octadecyl, N, N dihydroxyethyl ammonium chloride [lipid D0] or N-oleyl, N-octadecyl, N, N dihydroxyethyl ammonium chloride [lipid D1] or their salt, solvate, derivatives, or combination thereof; along with neutral/zwitter-ionic co-lipids mixed in different ratios.

In another embodiment present invention discloses a cationic lipid (CL) based composition comprising cationic lipid N, N di-octadecyl, N, N dihydroxyethyl ammonium chloride [lipid D0] or N-oleyl, N-octadecyl, N, N dihydroxyethyl ammonium chloride [lipid D1] or their salt, solvate, derivatives, or combination thereof; along with two or more neutral/zwitter-ionic co-lipids selected from steroid lipid, phospholipid, and phospholipid-polymer conjugate.

In one embodiment the cationic lipid (CL) based composition of the present invention further comprises a biologically active agent as Nucleic Acid (NA) derived from a living bio-organism and non-living bio-organism.

In one embodiment of the present invention cationic lipids N, N di-octadecyl, N, N dihydroxyethyl ammonium chloride [lipid D0] or N-oleyl, N-octadecyl, N, N dihydroxyethyl ammonium chloride [lipid D1] in combination with co-lipids and nucleic acid forms lipid/nucleic acid complex.

In one embodiment the present composition for biologically active nucleic acid (NA) delivery is formulated as Nanoparticle lipid formulation as a delivery system.

In another embodiment the present composition for biologically active nucleic acid (NA) delivery is formulated as Liposome/Liposomal formulation as a delivery system.

In another embodiment the present composition for biologically active nucleic acid (NA) delivery is formulated as lipoplex as a delivery system.

In one embodiment present invention discloses a cationic lipid (CL) based composition optionally comprising a pharmaceutically acceptable carrier or excipient.

In another aspect, the present invention is directed to a vaccine comprising the cationic lipid (CL) based composition and formulation of the present invention for the prophylaxis and/or treatment of virus mediated disease in a human subject.

In one aspect, the present invention discloses a vaccine formulation comprising nucleic acid as vaccine antigen for the prophylaxis and/or treatment of virus-mediated disease in a human subject.

In one embodiment present invention discloses a vaccine formulation comprising nucleic acid as vaccine antigen for the prophylaxis and/or treatment of virus-mediated disease in a human subject, comprising a Nucleic acid (NA) based biologically active antigenic agent and a cationic-based (CL) lipid composition of the present invention.

In one embodiment, the cationic-based (CL) lipid composition in the said vaccine formulation comprises cationic lipid N, N di-octadecyl, N, N dihydroxyethyl ammonium chloride [lipid D0] or N-oleyl, N-octadecyl, N, N dihydroxyethyl ammonium chloride [lipid D1] or their salt, solvate, derivatives, or combination thereof: along with neutral/zwitter-ionic co-lipids mixed in different ratios.

In another embodiment, the cationic-based (CL) lipid composition in the said vaccine formulation of the present invention comprises cationic lipid N, N di-octadecyl, N, N dihydroxyethyl ammonium chloride [lipid D0] or N-oleyl, N-octadecyl, N, N dihydroxyethyl ammonium chloride [lipid D1] or their salt, solvate, derivatives, or combination thereof: along with two or more neutral/zwitter-ionic co-lipids selected from steroid lipid, phospholipid, and phospholipid-polymer conjugate.

In one embodiment the present invention discloses a vaccine formulation for the prophylaxis and/or treatment of virus-mediated disease in a human subject comprising a Nucleic acid (NA) based biologically active antigenic agent selected from DNA, pDNA, RNA, mRNA, fragment, or combination thereof derived from virus.

In one embodiment the suitable lipid formulation in vaccine formulation is formulated as Nanoparticle lipid formulation as a delivery system.

In another embodiment the suitable lipid formulation in vaccine formulation is formulated as Liposome/Liposomal formulation as a delivery system.

In another embodiment the suitable lipid formulation in vaccine formulation is formulated as lipoplex as a delivery system.

In one aspect present invention discloses a method for preparation of a cationic lipid (CL) based composition.

In another aspect present invention discloses a method for preparation of vaccine formulation.

In yet another aspect, the present invention is a method for the treatment of a disease comprising the step of administering a therapeutically effective amount of cationic lipid (CL) based composition of the present invention to a patient in need of treatment thereof.

In one embodiment present invention discloses a method of prophylaxis, treatment, and management of virus-mediated disease by administering a vaccine formulation in a subject in the need thereof.

In another aspect of the present invention the immunogenicity of above said Lipid/nucleic acid complex is evaluated in animals for stability of lipid formulation in vivo following intramuscular injection.

### Cationic Lipid Based Composition:

The present invention discloses and provides a cationic lipid (CL) based composition comprising at least one cationic lipid of N, N di-octadecyl, N, N dihydroxyethyl ammonium chloride [lipid D0] or N-oleyl, N-octadecyl, N, N dihydroxyethyl ammonium chloride [lipid D1]. The lipid composition further comprises other lipids mixed in different ratios. Such composition is formulated in a suitable formulation which is used to deliver a biologically active agent as a candidate vaccine antigen such as nucleic acid derived vaccine antigen, in combination with one or more other lipid components and optionally one or more excipients.

The terms “salt” or “salts” used herein refers to an acid addition of a compound of the invention. “Salts” include in particular “pharmaceutically acceptable salts”. The term “pharmaceutically acceptable salts” refers to salts that retain the biological effectiveness and properties of the compounds of this invention and, which typically are not biologically or otherwise undesirable.

Pharmaceutically acceptable acid addition salts can be formed with inorganic acids and organic acids, e.g., acetate, aspartate, camphorsulfonate, chloride/hydrochloride, chlortheophyllonate, citrate, benzoate, besylate, bromide/hydrobromide, bicarbonate/carbonate, bisulfate/sulfate, ethandisulfonate, isethionate, lactate, lactobionate, laurylsulfate, malate, maleate, malonate, mandelate, mesylate, methylsulphate, naphthoate, napsylate, nicotinate, nitrate, fumarate, gluceptate, gluconate, glucuronate, hippurate, hydroiodide/iodide, phosphate/hydrogen phosphate/dihydrogen phosphate, polygalacturonate, propionate, stearate, succinate, octadecanoate, oleate, oxalate, palmitate, pamoate, subsalicylate, tartrate, tosylate and trifluoroacetate salts.

Inorganic acids from which salts can be derived include, for example, hydrochloric acid, hydrobromic acid, phosphoric acid, sulfuric acid, nitric acid and the like.

Organic acids from which salts can be derived include, for example, acetic acid, propionic acid, glycolic acid, toluenesulfonic acid, sulfosalicylic acid, fumaric acid, tartaric acid, citric acid, benzoic acid, mandelic acid, methanesulfonic acid, oxalic acid, maleic acid, malonic acid, succinic acid, ethanesulfonic acid, and the like.

The pharmaceutically acceptable salts of the present invention can be synthesized from a basic or acidic moiety, by conventional chemical methods.

The term “solvate” means a physical association of a compound of present invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like.

The active agent of the present invention may be a derivative of therapeutic agent. Therapeutic agent derivatives may be therapeutically active themselves or they may be prodrugs, which become active upon further modification. Thus, in one embodiment, a therapeutic agent derivative retains some or all of the therapeutic activity as compared to the unmodified agent, while in another embodiment, a therapeutic agent derivative lacks therapeutic activity.

Expressions “vaccine”, “vaccine formulation”, “vaccine composition” invariably used throughout the description and drawings will have the same meaning as a substance used to stimulate the production of antibodies and provide immunity against one or several diseases. The vaccine provides prophylaxis and treatment of various infections which is capable of conferring immunity against such infections.

The term “antigen” means any substance that causes the body to make an immune response against that substance. Antigens include toxins, chemicals, bacteria, viruses, or other substances that come from outside the body. Vaccines are examples of antigens in an immunogenic form, which are intentionally administered to a recipient to induce the memory function of the adaptive immune system towards antigens of the pathogen invading that recipient.

The term “composition” or “pharmaceutical composition” means a composition containing one or more drugs or prodrugs or active ingredient, along with other excipients.

The term “dose” or “dosage” means the size or amount of a dose of a medicine or drug.

The present invention discloses two cationic lipids N, N di-octadecyl, N, N dihydroxyethyl ammonium chloride [lipid D0] or N-oleyl, N-octadecyl, N, N dihydroxyethyl ammonium chloride [lipid D1]

According to the present invention, a cationic lipid (CL) based composition for Nucleic Acid (NA) delivery comprises cationic lipid N, N di-octadecyl, N, N dihydroxyethyl ammonium chloride [lipid D0] or N-oleyl, N-octadecyl, N, N dihydroxyethyl ammonium chloride [lipid D1]; or their salt, solvate, derivatives, or combination thereof.

Present invention discloses a cationic lipid (CL) based composition for Nucleic Acid (NA) delivery comprises cationic lipid N, N di-octadecyl, N, N dihydroxyethyl ammonium chloride [lipid D0] or N-oleyl, N-octadecyl, N, N dihydroxyethyl ammonium chloride [lipid D1] in combination with neutral/zwitter-ionic co-lipids.

These co-lipids are selected from steroid lipid, phospholipid, phospholipid-polymer conjugate or combination thereof.

The steroid lipid which may be sterol may include but not limited to Cholesterol, ergosterol, stigmasterol, sitosterol, campesterol, stigmastanol like phytosterols and other steroids, such as dexamethasone, prednisolone, triamcinolone and/or mixture thereof.

Preferably sterol is cholesterol.

The phospholipid may include but not limited to Dioleoyl phosphatidylethanolamine [DOPE], 1,2-Dioleoyl-sn-Glycero-3-Phosphocholine [DOPC], 1,2-Distearoyl-sn-glycero-3-phosphoethanolamine [DSPE], 1,2-Distearoyl-sn-glycero-3-phosphocholine [DSPC], 1,2-Dimyristoyl-sn-glycero-3-phosphoethanolamine [DMPE], 1,2-Dimyristoyl-sn-glycero-3-phosphocholine [DMPC], 1,2-Dipalmitoyl-sn-glycero-3-phosphoethanolamine [DPPE], 1,2-Dipalmitoyl-sn-glycero-3-phosphocholone [DPPC], or mixture thereof.

Preferably phospholipid is DOPE and/or DOPC: most preferably phospholipid is DOPE.

The phospholipid-polymer conjugate may include but not limited to conjugates prepared by conjugation of a suitable phospholipid selected from Dioleoyl phosphatidylethanolamine [DOPE], 1,2-Distearoyl-sn-glycero-3-phosphoethanolamine [DSPE], 1,2-Dimyristoyl-sn-glycero-3-phosphoethanolamine [DMPE], 1,2-Dipalmitoyl-sn-glycero-3-phosphoethanolamin [DPPE] with a suitable polymer which is selected from polyethylene-glycol [PEG] of 2000, 5000, 8000 Average molecular weight.

Preferably the Phospholipid-Polymer conjugate may be DSPE-PEG with varied PEG average molecular weight: most preferably the Phospholipid-Polymer conjugate is DSPE-PEG (2000).

The cationic lipid (CL) based composition of the present invention optionally comprises a pharmaceutically acceptable carrier or excipient.

Accordingly, present invention discloses a cationic lipid (CL) based composition for nucleic acid (NA) delivery, wherein the CL-based composition comprises:
(a) Cationic lipid: N, N di-octadecyl, N, N dihydroxyethyl ammonium chloride [lipid D0] or N-oleyl, N-octadecyl, N, N dihydroxyethyl ammonium chloride [lipid D1]; or its salt, solvate, derivatives, or combination thereof:
(b) combination of Neutral/Zwitter-ionic co-lipids which comprises two or more lipids selected from:
   (b1) steroid lipid which may be sterol and is selected from Cholesterol, ergosterol, stigmasterol, sitosterol, campesterol, stigmastanol like phytosterols and other steroids such as dexamethasone, prednisolone, triamcinolone and/or mixture thereof:
   (b2) phospholipid which may be selected from Dioleoyl phosphatidylethanolamine [DOPE], 1,2-Dioleoyl-sn-Glycero-3-Phosphocholine [DOPC], 1,2-Distearoyl-sn-glycero-3-phosphoethanolamine [DSPE], 1,2-Distearoyl-sn-glycero-3-phosphocholine [DSPC], 1,2-Dimyristoyl-sn-glycero-3-phosphoethanolamine [DMPE], 1,2-Dimyristoyl-sn-glycero-3-phosphocholine [DMPC], 1,2-Dipalmitoyl-sn-glycero-3-phosphoethanolamine [DPPE], 1,2-Dipalmitoyl-sn-glycero-3-phosphocholone [DPPC] or mixture thereof, preferably DOPE and/or DOPC; and
   (b3) a phospholipid-polymer conjugate which may be selected from conjugates prepared by conjugation of a suitable phospholipid selected from Dioleoyl phosphatidylethanolamine [DOPE], 1,2-Distearoyl-sn-glycero-3-phosphoethanolamin [DSPE], 1,2-Dimyristoyl-sn-glycero-3-phosphoethanolamine [DMPE], 1,2-Dipalmitoyl-sn-glycero-3-phosphoethanolamin [DPPE], with a suitable polymer which is selected from polyethylene-glycol [PEG] of 2000, 5000, 8000 Average molecular weight, and
(c) optionally, one or more pharmaceutically acceptable excipient.

In one embodiment, cationic lipid (CL) based composition for nucleic acid (NA) delivery of the present invention, wherein the steroid lipid is cholesterol.

In another embodiment, the cationic lipid (CL) based composition for nucleic acid (NA) delivery of the present invention, wherein the phospholipid (b2) is DOPE and/or DOPC: most preferably phospholipid (b2) is DOPE.

In another embodiment, the cationic lipid (CL) based composition for nucleic acid (NA) delivery of the present invention, wherein the Phospholipid-Polymer conjugate (b3) is DSPE-PEG with varied PEG average molecular weight: most preferably the Phospholipid-Polymer conjugate is DSPE-PEG (2000).

In one embodiment of the present invention, the cationic lipid N, N di-octadecyl, N, N dihydroxyethyl ammonium chloride [lipid D0] or N-oleyl, N-octadecyl, N, N dihydroxyethyl ammonium chloride [lipid D1] is with or without Phospholipid-Polymer conjugate.

In one preferred embodiment present invention discloses a cationic lipid (CL) based composition for nucleic acid (NA) delivery, wherein the composition comprises:
(a) cationic lipid N, N di-octadecyl, N, N dihydroxyethyl ammonium chloride [lipid D0] or N-oleyl, N-octadecyl, N, N dihydroxyethyl ammonium chloride [lipid D1];
(b) neutral/zwitter-ionic co-lipids selected from (b1) Cholesterol, (b2) Dioleoylphosphatidylethanolamine [DOPE], with or without Phospholipid-Polymer conjugate (b3) 1,2-distearoyl-sn-glycero-3-phosphoethanolamine-N-[amino(polyethylene glycol)-2000 [DSPE-PEG]; and
(c) optionally, one or more pharmaceutically acceptable excipient.

In one embodiment present invention discloses the cationic lipid-based composition for nucleic acid (NA) delivery comprising: N, N di-octadecyl, N, N dihydroxyethyl ammonium chloride [lipid D0] or N-oleyl, N-octadecyl, N, N dihydroxyethyl ammonium chloride [lipid D1]; in combination with 1,2-Dioleoyl-sn-glycero-3-phosphoethanolamine [DOPE], Cholesterol and 1,2-distearoyl-sn-glycero-3-phosphoethanolamine-N-[amino(polyethylene glycol)-2000 [DSPE-PEG].

In such preferred embodiment present invention discloses the cationic lipid-based composition for nucleic acid (NA) delivery comprising: N-oleyl, N-octadecyl, N, N dihydroxyethyl ammonium chloride [lipid D1], 1,2-Dioleoyl-sn-glycero-3-phosphoethanolamine [DOPE], Cholesterol and 1,2-distearoyl-sn-glycero-3-phosphoethanolamine-N-[amino(polyethylene glycol)-2000 [DSPE-PEG].

In the said embodiment the mole ratio of N-oleyl, N-octadecyl, N, N dihydroxyethyl ammonium chloride [lipid D1], 1,2-Dioleoyl-sn-glycero-3-phosphoethanolamine [DOPE], Cholesterol and 1,2-distearoyl-sn-glycero-3-phosphoethanolamine-N-[amino(polyethylene glycol)-2000 [DSPE-PEG] is 1:1:1:0.03.

In one embodiment present invention discloses the cationic lipid-based composition for nucleic acid (NA) delivery comprising: N, N di-octadecyl, N, N dihydroxyethyl ammonium chloride [lipid D0] or N-oleyl, N-octadecyl, N, N dihydroxyethyl ammonium chloride [lipid D1]; in combination with 1,2-Dioleoyl-sn-glycero-3-phosphoethanolamine [DOPE] and Cholesterol.

In such preferred embodiment present invention discloses the cationic lipid-based composition for nucleic acid (NA) delivery comprising: N-oleyl, N-octadecyl, N, N dihydroxyethyl ammonium chloride [lipid D1], 1,2-Dioleoyl-sn-glycero-3-phosphoethanolamine [DOPE] and Cholesterol.

In the said embodiment the mole ratio of N-oleyl, N-octadecyl, N, N dihydroxyethyl ammonium chloride [lipid D1], 1,2-Dioleoyl-sn-glycero-3-phosphoethanolamine [DOPE] and Cholesterol 1:1:1.

### Biologically Active Agent

The cationic lipid-based composition of the present invention further comprises one or more biologically active agents including, but not limited to hormones, antibodies, cholesterol, peptides, proteins, nucleic acid etc.

In one embodiment the cationic lipid-based composition of the present invention comprising a biologically active agent, wherein the biologically active agent is a nucleic acid.

In the present invention, the Nucleic Acid (NA) is a biologically active agent which is delivered to a subject in need thereof using the present CL composition, wherein the Nucleic Acid (NA) is selected from ribonucleic acid (RNA), messenger RNA (mRNA), Deoxyribonucleic acid (DNA), plasmid DNA (pDNA), fragment of RNA, mRNA, DNA, pDNA, miRNA or any chimeric or fusion thereof.

The said Nucleic Acid (NA) is derived from a living bio-organism and non-living bio-organism.

The biologically active agent i.e. Nucleic Acid (NA) is derived from a virus which may be selected from Coronaviridae, Retroviridae, Reoviridae, Togaviridae, Filoviridae,

Flavirideae, Papillomaviridae, Plasmodiidae, Hepadnaviridae, Picornaviridae, Caliciviridae, Orthomyxoviridae, Adenoviridae, Rhabdoviridae, Paramyxoviridae, Orthomyxoviridae, Nodaviridae etc.

According to the present invention the biologically active agent is a DNA, pDNA, RNA, mRNA derived from the virus selected from Coronaviridae, Retroviridae, Reoviridae, Togaviridae, Filoviridae, Flavirideae, Papillomaviridae, Plasmodiidae, Hepadnaviridae, Picornaviridae, Caliciviridae, Orthomyxoviridae, Adenoviridae, Rhabdoviridae, Paramyxoviridae, Orthomyxoviridae and Nodaviridae.

In one of the preferred embodiments, the biologically active agent is a DNA or mRNA derived from above listed virus.

In the present invention N, N di-octadecyl, N, N dihydroxyethyl ammonium chloride [lipid D0] or N-oleyl, N-octadecyl, N, N dihydroxyethyl ammonium chloride [lipid D1] in combination with co-lipids as described above and nucleic acid forms lipid/nucleic acid complex.

### Forms of Lipid Composition:

The cationic lipid (CL) based composition of the present invention for biologically active Nucleic Acid (NA) delivery is formulated in a suitable lipid formulation preparation as a delivery system with or without one or more pharmaceutically acceptable excipient, wherein the suitable lipid formulation may be selected from:
Nanoparticle formulation,
Lipid/Liposome/Liposomal formulation,
Lipoplex formulation,
Lipid/polymer emulsion formulation.

In one embodiment the lipid composition is in the form of a liposome.

A liposome is a vesicle that can be filled with drugs or actives and can be used to deliver these drugs or actives at the target sites. These liposomes are made up of same material as that of cell membrane material such as phospholipids.

Liposomes entrap nucleic acid by one of two mechanisms which have resulted in their classification as either cationic liposomes or pH-sensitive liposomes. Cationic liposomes are positively charged liposomes which interact with the negatively charged nucleic acid molecules to form a stable complex. pH sensitive liposomes are lipid compositions that can be destabilized when the external pH is changed i.e. from neutral or slightly alkaline pH to an acidic

There are three types of liposomes: MLV (Multilamellar vesicles), SUV (Small unilamellar vesicles) and LUV (Large unilamellar vesicles). MLVs have multiple bilayers in each vesicle, forming several separate aqueous compartments. SUVs and LUVs have a single bilayer encapsulating an aqueous core.

Liposomes are used for drug delivery due to their unique properties. They can contain a wide variety of hydrophilic and hydrophobic diagnostic or therapeutic agents, providing a larger drug payload per particle and protecting the encapsulated agents from metabolic processes.

The cationic lipid (CL) based composition of the present invention forms a Liposomal formulation for nucleic acid (NA) delivery for nucleic acid-based vaccine therapeutics.

In another embodiment the lipid composition is in the form of a lipid nanoparticle (LNP).

Lipid nanoparticles are spherical vesicles made of ionizable lipids, which are positively charged at low pH enabling nucleic acid complexation and neutral at physiological pH, reducing potential toxic effects, as compared with positively charged lipids, such as liposomes.

Lipid-based nanoparticle (LBNP) systems represent one of the most promising colloidal carriers for bioactive organic molecules. These nanoparticles can transport hydrophobic and hydrophilic molecules, display very low or no toxicity, and increase the time of drug action by means of a prolonged half-life and a controlled release of the drug.

Different methods for loading biologically active agents into lipid compositions, such as lipid nanoparticles are available in the art. The nucleic acid molecule is generally of a size that it can be encapsulated in a lipid nanoparticle of the present invention.

The cationic lipid (CL) based composition of the present invention forms a Lipid nanoparticle formulation for nucleic acid (NA) delivery for nucleic acid-based vaccine therapeutics.

In another embodiment the lipid composition is in the form of a lipoplexes

Lipoplexes are complexes formed among plasmid nucleic acid and cationic lipids. Lipoplexes are liposome-based formulations that are formed by electrostatic interaction of cationic liposomes with anionic nucleic acids. The lipids spontaneously form aggregates and carry surface positive charges that make electrostatic complexation with nucleic acids, which are negatively charged. Formed lipoplexes possess distinct internal arrangements of molecules that arise due to the transformation from liposomal structure into compact RNA-lipoplexes.

Nucleic acids entrapped in lipids (lipoplexes) are attractive vehicles for the delivery of nucleic acids because they provide nuclease resistance and for larger nucleic acids such mRNA provides condensation, which facilitate cellular uptake.

The cationic lipid (CL) based composition of the present invention is in the form of Lipoplex for nucleic acid (NA) delivery for nucleic acid-based vaccine therapeutics.

In another embodiment the lipid composition is in the form of Lipid/polymer emulsion formulation.

The lipid/polymer emulsion formulation comprises lipid components, a nucleic acid and an aqueous carrier. The said emulsion formulation can be used in manufacturing of a medicament for the treatment of a condition, wherein the treatment comprises the delivery of a nucleic acid to the cells of the human or animal.

The cationic lipid (CL) based composition of the present invention forms a Lipid/polymer emulsion formulation for nucleic acid (NA) delivery for nucleic acid-based vaccine therapeutics.

In one of the preferred embodiments, the cationic lipid (CL) based composition of the present invention is formulated as a cationic lipid (CL) based formulation formulated along with the biologically active Nucleic Acid (NA) to provide a pharmaceutical formulation with or without one or more pharmaceutically acceptable excipient, to administer in a subject in need thereof, wherein the pharmaceutical formulation comprises:
a Lipid-Nanoparticle (LNP) formulation or a Liposomal formulation—encapsulating the biologically active agent, or
a Lipoplex formulation—formed by complexion of Liposomal preparation with the biologically active agent.

The cationic lipid (CL) based composition of the present invention is a vaccine formulation for delivery of biologically active agent which comprises:
a complex of Lipid composition+DNA/pDNA (DNA-Lipoplex), or
a complex of Lipid composition+RNA/mRNA complex (RNA-Lipoplex).

### Pharmaceutically Acceptable Excipients

The cationic lipid (CL) based composition of the present invention further comprises of pharmaceutically acceptable excipients.

The composition comprising a cationic lipid (CL) formulated along with the biologically active Nucleic Acid (NA) of the present invention generally may comprise and/or formulated with or without one or more pharmaceutically acceptable excipient(s), suitable for composition or formulation to be administered in mammals through various routes of administration in suitable concentration, which may be selected from group comprising of adjuvants, buffers, diluents, lubricants, binders, stabilizers, preservatives, disintegrants, absorbents, colorants surfactants, residuals, or combination thereof.

The pharmaceutically acceptable excipients may include but not limited to:
Buffer such as Tris buffer, phosphate buffer, borate buffer or combination/mixture thereof:
Stabilizers in an appropriate concentration to impart stability to the vaccine composition and/or formulation. Examples of such suitable stabilizer(s) that may be included in the composition comprises Magnesium chloride (MgCl), Polysorbate-80 (P-80) or like:
Diluents such as lactose, dextrose, sucrose, mannitol, sorbitol, cellulose and/or glycine. The diluent or carrier may be an inert solvent or a medium for liquid solution or dispersion which may be selected from water, polyol (for example, glycerol, and the like), and suitable mixtures thereof:
Lubricants such as. silica, talcum, stearic acid, its magnesium or calcium salt and/or polyethyleneglycol:
Binders such as magnesium aluminum silicate, starch paste, gelatin, tragacanth, methylcellulose, sodium carboxymethylcellulose and/or polyvinylpyrrolidone:
Disintegrants such as starches, agar, alginic acid or its sodium salt, or effervescent mixtures:
Absorbents, colorants, flavors and/or sweeteners; and/or
Immune boosting co-excipients like terpenes and terpenoid molecules such as linalool, myrcene, squalene, beta pinene, caryophyllene, limonene etc.

### Method for Preparation of Cationic Lipid (CL) Based Formulation:

A method for preparation of a cationic lipid (CL) based composition, comprising the steps of:
a) taking CL and other co-lipids and excipients first in chloroform and mixing thoroughly in a vial:
b) drying off chloroform by thin layer of N₂ flow, followed by keeping the vial at high vacuum; and
c) dissolving the residual lipid mixture in minimal ethanol followed by rapidly injecting the ethanolic lipid mixture in the suitable buffer solution, typically of pH 7.4, to make the CL based formulation in room temperature.

### Immunogenicity:

Present invention further describes immunogenicity of cationic lipid (CL) based composition and formulation of the present invention which is evaluated in animals for stability of lipid formulation in vivo following intramuscular injection.

Most formulations are stable over time and showed varied degree of DNA complexation. The formulations with maximum nucleic acid complexation ability with comparatively lesser amount of cationic lipid are further chosen for plasmid and mRNA transfection efficiencies. The formulations exhibiting low to high level of pDNA/mRNA transfection in HEK293 cells are further used to deliver luciferase encoding mRNA construct (Luc-mRNA) in mouse. The lipid/mRNA complexes are delivered to femoral muscle of BalbC albino mice and luciferase activities due to the injection of respective formulations were detected at various time points. In addition stability of lipid formulation in vivo following intramuscular injection have been examined.

The class of the delivery system is composed of the concoctions of a cationic lipid, D1 or D0 and other neutral/zwitter-ionic co-lipids with or without PEG-lipid.

The formulations enabled efficient in vivo transfection of mRNAs at a much lower +/−charge ratio (N/P) in comparison to existing, commercial, cationic-ionizable lipid-based non-viral formulations, which generally work at higher N/P ratio. The present invention shows that the formulation is able to perform in vivo transfection at a N/P charge ratio of 1:1 and above, preferably 1:1, whereas the existing commercial formulations use N/P charge ratio of ≥6:1.

Accordingly present invention discloses a cationic lipid (CL) based composition, wherein the formulation performs in vivo transfection at a N/P charge ratio (Lipid: Nucleic acid) of 1:1 to 4:1.

Preferably the N/P charge ratio is 1:1 or 2:1 or 3:1 or 4:1.

Moreover, the lipid/RNA complex (lipoplex) is stable at room temperature for at least 30 days without any compromise in its transfection output, thus enabling a situation tenable for long time, low-cost storage and transportation. In contrast, the lipoplex formed from commercial formulations need sub-zero temperature for preservation toward maintaining its activity thus demand higher storage cost.

The lipoplex formulation following intramuscular injection in mice continued to exhibit the expression of transgene for at least up to 15 days whereas the injected lipid (using a DiR dye) was found to be almost completely removed from mouse body by 5th day indicating that the lipid-mediated toxicity, if any, due to its longer body exposure may be minimized.

Accordingly, the cationic lipid (CL) based composition, or the cationic lipid (CL) based formulation of the present invention including the vaccine formulation, wherein the composition and/or formulation minimizes lipid-mediated toxicity in a subject administered with, by minimizing the amount of cationic-lipid required in the composition/formulation.

### Vaccine Formulation:

In another aspect, present invention is directed towards the vaccine formulation comprising the cationic lipid (CL) based composition or the cationic lipid (CL) based formulation of the present invention for the prophylaxis and/or treatment of virus mediated disease in a human subject.

Accordingly present invention discloses a vaccine formulation comprising biologically active agent as vaccine antigen along with a cationic lipid (CL) based composition for the prophylaxis and/or treatment of disease in a human subject.

### Cationic Lipid (CL) Based Composition:

The present invention discloses the Vaccine formulation with above-described novel cationic lipid (CL) based composition or the cationic lipid (CL) based formulation.

According to the present invention, a cationic lipid (CL) based composition for Nucleic Acid (NA) delivery comprises cationic lipid N, N di-octadecyl, N, N dihydroxyethyl ammonium chloride [lipid D0] or N-oleyl, N-octadecyl, N, N dihydroxyethyl ammonium chloride [lipid D1]; or their salt, solvate, derivatives, or combination thereof. Herein salt, solvate, derivatives are as defined above.

Present invention discloses a cationic lipid (CL) based composition for Nucleic Acid (NA) delivery comprises cationic lipid N, N di-octadecyl, N, N dihydroxyethyl ammonium chloride [lipid D0] or N-oleyl, N-octadecyl, N, N dihydroxyethyl ammonium chloride [lipid D1] in combination with neutral/zwitter-ionic co-lipids.

These co-lipids are selected from steroid lipid, phospholipid, phospholipid-polymer conjugate or combination thereof.

The steroid lipid which may be sterol may include but not limited to Cholesterol, ergosterol, stigmasterol, sitosterol, campesterol, stigmastanol like phytosterols and other steroids, such as dexamethasone, prednisolone, triamcinolone and/or mixture thereof.

In one embodiment, the vaccine composition comprising cationic lipid (CL) based composition for nucleic acid (NA) delivery, wherein the steroid lipid is cholesterol.

The phospholipid may include but not limited to Dioleoyl phosphatidylethanolamine [DOPE], 1,2-Dioleoyl-sn-Glycero-3-Phosphocholine [DOPC], 1,2-Distearoyl-sn-glycero-3-phosphoethanolamine [DSPE], 1,2-Distearoyl-sn-glycero-3-phosphocholine [DSPC], 1,2-Dimyristoyl-sn-glycero-3-phosphoethanolamine [DMPE], 1,2-Dimyristoyl-sn-glycero-3-phosphocholine [DMPC], 1,2-Dipalmitoyl-sn-glycero-3-phosphoethanolamine [DPPE], 1,2-Dipalmitoyl-sn-glycero-3-phosphocholone [DPPC] or mixture thereof.

In another embodiment, the vaccine composition comprising cationic lipid (CL) based composition for nucleic acid (NA) delivery of the present invention, wherein the phospholipid (b2) is DOPE and/or DOPC: most preferably phospholipid (b2) is DOPE.

The phospholipid-polymer conjugate may include but not limited to conjugates prepared by conjugation of a suitable phospholipid selected from Dioleoyl phosphatidylethanolamine [DOPE], 1,2-Distearoyl-sn-glycero-3-phosphoethanolamine [DSPE], 1,2-Dimyristoyl-sn-glycero-3-phosphoethanolamine [DMPE], 1,2-Dipalmitoyl-sn-glycero-3-phosphoethanolamin [DPPE] with a suitable polymer which is selected from polyethylene-glycol [PEG] of 2000, 5000, 8000 Average molecular weight.

In another embodiment, the vaccine composition comprising cationic lipid (CL) based composition for nucleic acid (NA) delivery of the present invention, wherein the Phospholipid-Polymer conjugate (b3) is DSPE-PEG with varied PEG average molecular weight: most preferably the Phospholipid-Polymer conjugate is DSPE-PEG (2000).

In one embodiment of the present invention, the vaccine formulation comprising the cationic lipid N, N di-octadecyl, N, N dihydroxyethyl ammonium chloride [lipid D0] or N-oleyl, N-octadecyl, N, N dihydroxyethyl ammonium chloride [lipid D1] is with or without Phospholipid-Polymer conjugate.

The cationic lipid (CL) based composition of the present invention optionally comprises a pharmaceutically acceptable carrier or excipient. The pharmaceutically acceptable carrier or excipient are as defined above.

### Biologically Active Agent:

The present invention discloses the Vaccine formulation with above-described novel cationic lipid (CL) based composition or the cationic lipid (CL) based formulation formulated with biologically active agents not limited to hormones, antibodies, cholesterol, peptides, proteins, nucleic acid etc.

In one embodiment the Vaccine formulation of the present invention comprises a biologically active agent, wherein the biologically active agent is a nucleic acid.

According to the present invention the said nucleic acid (NA) is selected from ribonucleic acid (RNA), messenger RNA (mRNA), Deoxyribonucleic acid (DNA), plasmid DNA (pDNA), fragment of RNA, mRNA, DNA, pDNA, miRNA or any chimeric or fusion thereof.

The said nucleic acid (NA) is derived from a living bio-organism and non-living bio-organism.

The biologically active agent i.e. Nucleic Acid (NA) is derived from a virus which may be selected from the group comprising Coronaviridae, Retroviridae, Reoviridae, Togaviridae, Filoviridae, Flavirideae, Papillomaviridae, Plasmodiidae, Hepadnaviridae, Picornaviridae, Caliciviridae, Orthomyxoviridae, Adenoviridae, Rhabdoviridae, Paramyxoviridae, Orthomyxoviridae, Nodaviridae etc.

According to the present invention the biologically active agent is ribonucleic acid (RNA), messenger RNA (mRNA), Deoxyribonucleic acid (DNA), plasmid DNA (pDNA), fragment of RNA, mRNA, DNA, pDNA, miRNA or any chimeric or fusion thereof is derived from a virus from Coronaviridae, Retroviridae, Reoviridae, Togaviridae, Filoviridae, Flavirideae, Papillomaviridae, Plasmodiidae, Hepadnaviridae, Picornaviridae, Caliciviridae, Orthomyxoviridae, Adenoviridae, Rhabdoviridae, Paramyxoviridae, Orthomyxoviridae, Nodaviridae etc.

In one of the preferred embodiments, the vaccine formulation comprises DNA, pDNA, RNA, mRNA or combination thereof derived from virus.

### Vaccine Formulation of Present Invention:

Accordingly present invention discloses a vaccine formulation comprising nucleic acid as vaccine antigen for the prophylaxis and/or treatment of virus-mediated disease in a human subject, wherein the vaccine comprises:
a Nucleic acid (NA) based biologically active vaccine antigen selected from DNA, pDNA, RNA, mRNA, fragment, or combination thereof derived from virus:
a cationic lipid (CL) based composition which comprises
   a. Cationic lipid: N, N di-octadecyl, N, N dihydroxyethyl ammonium chloride [lipid D0] or N-oleyl, N-octadecyl, N, N dihydroxyethyl ammonium chloride [lipid D1]; or its salt, solvate, derivatives, or combination thereof; and
   b. combination of Neutral/Zwitter-ionic co-lipids which comprises two or more lipids selected from:
      (b1) steroid lipid which may be sterol and is selected from Cholesterol, ergosterol, stigmasterol, sitosterol, campesterol, stigmastanol like phytosterols and other steroids such as dexamethasone, prednisolone, triamcinolone and/or mixture thereof:
      (b2) phospholipid which may be selected from Dioleoyl phosphatidylethanolamine [DOPE], 1,2-Dioleoyl-sn-Glycero-3-Phosphocholine [DOPC], 1,2-Distearoyl-sn-glycero-3-phosphoethanolamine [DSPE], 1,2-Distearoyl-sn-glycero-3-phosphocholine [DSPC], 1,2-Dimyristoyl-sn-glycero-3-phosphoethanolamine [DMPE], 1,2-Dimyristoyl-sn-glycero-3-phosphocholine [DMPC], 1,2-Dipalmitoyl-sn-glycero-3-phosphoethanolamine [DPPE], 1,2-Dipalmitoyl-sn-glycero-3-phosphocholone [DPPC] or mixture thereof, preferably DOPE and/or DOPC; and
      (b3) a phospholipid-polymer conjugate which may be selected from conjugates prepared by conjugation of a suitable phospholipid selected from Dioleoyl phosphatidylethanolamine [DOPE], 1,2-Distearoyl-sn-glycero-3-phosphoethanolamin [DSPE], 1,2-Dimyristoyl-sn-glycero-3-phosphoethanolamine [DMPE], 1,2-Dipalmitoyl-sn-glycero-3-phosphoethanolamin [DPPE], with a suitable polymer which is selected from polyethylene-glycol [PEG] of 2000, 5000, 8000 Average molecular weight, and
   c. optionally, one or more pharmaceutically acceptable excipient.

In the said vaccine formulation the vaccine antigen DNA, pDNA, RNA, mRNA, fragment, or combination thereof are derived from virus selected from Coronaviridae,

Retroviridae, Reoviridae, Togaviridae, Filoviridae, Flavirideae, Papillomaviridae, Plasmodiidae, Hepadnaviridae, Picornaviridae, Caliciviridae, Orthomyxoviridae, Adenoviridae, Rhabdoviridae, Paramyxoviridae, Orthomyxoviridae or Nodaviridae.

In one embodiment of the present invention, the vaccine formulation comprising the cationic lipid N, N di-octadecyl, N, N dihydroxyethyl ammonium chloride [lipid D0] or N-oleyl, N-octadecyl, N, N dihydroxyethyl ammonium chloride [lipid D1] is with or without Phospholipid-Polymer conjugate.

In one of the preferred embodiments, present invention discloses a vaccine formulation, wherein the vaccine antigen is mRNA formulated with a suitable lipid formulation for delivery of active antigenic agent which comprises:
(a) cationic lipid N-oleyl, N-octadecyl, N, N dihydroxyethyl ammonium chloride [lipid-D1]:
(b) neutral/zwitter-ionic co-lipids selected from (b1) Cholesterol, (b2) Dioleoylphosphatidylethanolamine [DOPE], with or without Phospholipid-Polymer conjugate (b3) 1,2-distearoyl-sn-glycero-3-phosphoethanolamine-N-[amino(polyethylene glycol)-2000 [DSPE-PEG]; and
(c) optionally, one or more pharmaceutically acceptable excipient.

In one embodiment the mole ratio of N-oleyl, N-octadecyl, N, N dihydroxyethyl ammonium chloride [Lipid-D1], Cholesterol, 1,2-Dioleoyl-sn-glycero-3-phosphoethanolamine [DOPE] in the composition of the vaccine formulation is 1:1:1.

In another embodiment, the mole ratio of N-oleyl, N-octadecyl, N, N dihydroxyethyl ammonium chloride [Lipid-D1], Cholesterol, 1,2-Dioleoyl-sn-glycero-3-phosphoethanolamine [DOPE], and 1,2-distearoyl-sn-glycero-3-phosphoethanolamine-N-[amino(polyethylene glycol)-2000 [DSPE-PEG] in the composition of the vaccine formulation is 1:1:1:0.03.

The said vaccine formulation may be formulated with or without one or more pharmaceutically acceptable excipient, wherein the pharmaceutically acceptable excipients are selected from buffers, adjuvants, diluents, lubricants, binders, stabilizers, preservatives, disintegrants, absorbents, colorants surfactants, flavors, sweeteners. residuals, immune boosting co-excipients or combination thereof.

In the said vaccine composition, the suitable lipid formulation may be selected from:
Nanoparticle formulation,
Lipid/Liposome/Liposomal formulation,
Lipoplex formulation,
Lipid/polymer emulsion formulation.

In one embodiment the cationic lipid (CL) based formulation in the said vaccine formulation of the present invention forms a Liposomal formulation.

In another embodiment the cationic lipid (CL) based formulation in the said vaccine formulation of the present invention forms a Lipid nanoparticle formulation.

In yet another embodiment the cationic lipid (CL) based formulation in the said vaccine formulation of the present invention forms a Lipoplex.

The vaccine formulation of the present invention, wherein the suitable lipid formulation is selected from:
a Lipid-Nanoparticle (LNP) formulation or a Liposomal formulation—encapsulating the mRNA,
a Lipoplex formulation—formed by complexion of Liposomal preparation with mRNA.

The said vaccine formulation performs in vivo transfection at a N/P charge ratio (Lipid: mRNA) of 1:1 to 4:1.

Preferably, the N/P charge ratio is 1:1 or 2:1 or 3:1 or 4:1.

The vaccine formulation of the present invention is stable at room temperature for at least 30 days.

The vaccine formulation of the present invention minimizes lipid-mediated toxicity in a subject administered with vaccine in need thereof, by minimizing the amount of cationic-lipid required in the composition/formulation of the vaccine.

### Method for Preparation of Vaccine:

A method for preparation of vaccine formulation, comprising the steps of:
a) preparation of RNA construct by in vitro transcription process;
b) complexing the CL based formulation as describe above with RNA at a fixed mole ratio to make lipid/RNA complex or ‘lipoplex’;
c) obtaining the lipoplex as the vaccine solution and preserving it at room temperature or at 4° C.

### Forms of Vaccine Formulation:

The vaccine formulation of the present invention is in liquid, powder, lyophilized or suspension form.

The vaccine formulation of the invention may be prepared in various forms, e.g., for injection either as liquid solutions or suspensions.

The vaccine formulation of the invention may be lyophilized or in aqueous form, i.e. solutions or suspensions. Liquid formulations may advantageously be administered directly from their packaged form and are thus ideal for injection without the need for reconstitution in aqueous medium as otherwise required for lyophilized compositions of the invention.

Alternatively, vaccine compositions of the present invention may be lyophilized and reconstituted, e.g., using one of a multitude of methods for freeze drying well known in the art to form dry, regular shaped (e.g., spherical) particles, such as micropellets or microspheres.

### Administration of the Vaccine Formulation:

The vaccine formulation of the present invention can be administered to animals and humans through intranasal, intraperitoneal, oral, intramuscular, subcutaneous or intradermal routes to test the immunogenicity.

In one embodiment of the invention, the candidate vaccine can be administered either as a single dose or in two or more doses by intranasal, intraperitoneal, oral, intramuscular, subcutaneous or intradermal routes in animals and humans to elicit the immune response.

In another embodiment of the invention, the candidate vaccine can be administered followed by vaccination with another vaccine by intranasal, intraperitoneal, oral, intramuscular, subcutaneous or intradermal routes in animals and humans to elicit the immune response.

In another embodiment of the invention, assays for neutralizing antibody titers were conducted to check the neutralizing antibody levels against vaccine formulations of the present invention which has shown to elicit the high level of neutralizing antibodies.

In the preferred embodiment present invention discloses a method of prophylaxis, treatment, and management of virus-mediated disease by administering a vaccine formulation in a subject in the need thereof by intranasal, intraperitoneal, oral, intramuscular, subcutaneous or intradermal routes.

### Method of Treatment:

In another aspect the invention discloses a method of eliciting a protective immune response in mammals including humans comprising administering the vaccine composition of the present invention by any route comprising intramuscular, intradermal, subcutaneous, intravenous, oral, intranasal or transcutaneous routes. In preferred embodiment present invention discloses a method of prophylaxis, treatment, and management of virus-mediated disease by administering a vaccine formulation in a subject in the need thereof.

The vaccine composition of the invention may be administered by any method comprising needles and syringes including pre-filled syringes, microneedle patch, needle-free patch, inhalation and nasal sprays.

### Use of Cationic Lipid (CL) Based Composition of the Present Invention:

delivery of Nucleic acid:
minimizing toxicity of cationic lipid by reducing the effective amount of lipid required for efficient gene transfer:
in preparation of pharmaceutical formulation including vaccine formulation;
in prophylaxis, treatment, and management of diseases caused by living and non-living microorganism including bacteria and viruses; and
in making formulations for delivering drugs (small molecule and/or genetic) for familial or acquired diseases such as cancer.

Present invention provides a cationic lipid-based formulations for nucleic acid delivery with maximum nucleic acid complexation ability and with comparatively lesser amount of cationic lipid having higher stability.

### EXAMPLES

The above-described aspects and embodiments of the invention further be understood by following non-limiting examples and corresponding drawing figures.

### Example 1: Preparation of Lipids

### D1 Lipid: N-Oleyl, N-Octadecyl, N, N Dihydroxyethyl Ammonium Chloride

Synthesis: Oleyl amine reacted with 1-bromooctadecane in presence of potassium carbonate, refluxed in ethyl acetate, followed by column chromatographic separation. The product, i.e. the secondary amine Yield=25%; Secondary amine refluxed in neat chloroethanol in presence of limited amount of aq. NaOH, followed by column chromatography and finally by crystallization of final product D1 lipid gave a yield of ≥95%. Overall yield from both reactions=23-25%. The first step can be modified to get better yield.

### D0 Lipid: N, N Di-Octadecyl, N, N-Dihydroxyethyl Ammonium Chloride

Synthesis: same as above except that 1-amino octadecane was used instead of oleyl amine. Overall yield=23-25%.

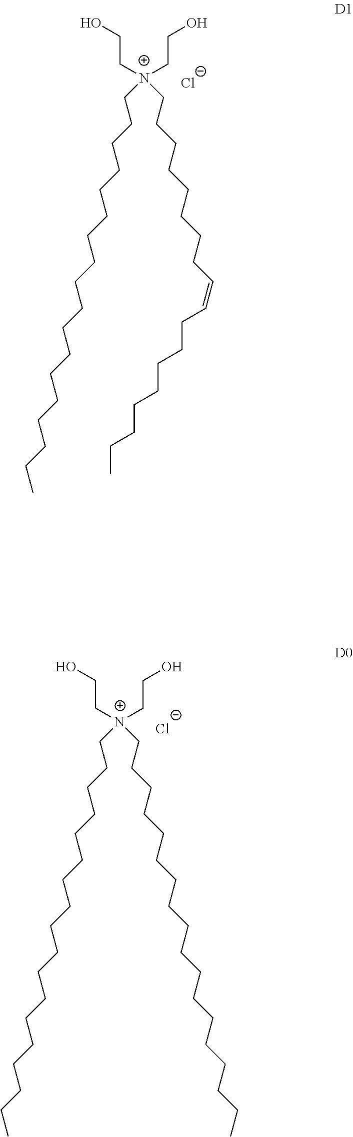 [SMILES: CCCCCCCC/C=C\CCCCCCCC[N+](CCO)(CCO)CCCCCCCCCCCCCCCCCC ; [Cl-] ; CCCCCCCCCCCCCCCCCC[N+](CCO)(CCO)CCCCCCCCCCCCCCCCCC ; [Cl-]]

### Example 2: Lipid Composition

The present invention discloses a cationic lipid (CL) based composition for nucleic acid (NA) delivery, wherein the CL-based composition comprises: Cationic lipid: N, N di-octadecyl, N, N dihydroxyethyl ammonium chloride [lipid D0] or N-oleyl, N-octadecyl, N, N dihydroxyethyl ammonium chloride [lipid D1] or their salt, solvate, derivatives, or combination thereof; in combination with Neutral/Zwitter-ionic co-lipids.

### General Lipid Composition:

The composition for cationic lipids for nucleic acid delivery may be prepared based on below shown general composition:

| | |
|---|---|
| | Concentration |
| Lipid composition | range (mM) |

Cationic lipid

| | | |
|---|---|---|
| N,N di-octadecyl, N,N dihydroxyethyl | 1 | mM |
| ammonium chloride [lipid D0] or N-oleyl, | | |
| N-octadecyl, N,N dihydroxyethyl | | |
| ammonium chloride [lipid D1] or their | | |
| salt, solvate, derivatives, or combination | | |
| thereof | | |

Neutral/Zwitter-ionic co-lipids

| | | |
|---|---|---|
| Cholesterol | 0.1-1 | mM |
| DOPC | 0.1-1 | mM |
| DOPE | 0.1-2 | mM |
| Dexamethasone | 0.01-1 | mM |
| DSPE-PEG | 0.01-0.05 | mM |

### Lipid Formulation:

18 different formulations were prepared encompassing the lipid D0 or D1. Following 18 formulations were prepared.

### Method: Ethanol Injection

Concentration: 1 mM (volume 1 mL) with respect to cationic lipid (D0 or D1)

| Formu- | | | Choles- | | | Dexa- | DSPE- |
|---|---|---|---|---|---|---|---|
| lations | D1 | D0 | terol | DOPC | DOPE | methasone | PEG |
| CLF 1 | | 1 | 1 | | 1 | | 0.03 |
| CLF 2 | 1 | | 1 | | 1 | | 0.03 |
| CLF 3 | 1 | | 0.5 | 1 | | 0.1 | 0.03 |
| CLF 4 | 1 | | 1 | 0.5 | | 0.1 | 0.03 |
| CLF 5 | 1 | | 0.75 | 0.75 | | 0.1 | 0.03 |
| CLF 6 | 1 | | 0.7 | 0.8 | | 0.1 | 0.03 |
| CLF 7 | 1 | | 0.5 | 1 | | | 0.03 |
| CLF 8 | 1 | | 1 | 0.5 | | | 0.03 |
| CLF 9 | 1 | | 0.75 | 0.75 | | | 0.03 |
| CLF 10 | 1 | | 0.7 | 0.8 | | | 0.03 |
| CLF 11 | | 1 | 0.5 | 1 | | | 0.03 |
| CLF 12 | | 1 | 1 | 0.5 | | | 0.03 |
| CLF 13 | | 1 | 0.5 | 1 | | 0.1 | 0.03 |
| CLF 14 | | 1 | 1 | 0.5 | | 0.1 | 0.03 |
| CLF 15 | | 1 | 1 | | | | |
| CLF 16 | 1 | | 1 | | | | |
| CLF 17 | | 1 | 1 | | 1 | | |
| CLF 18 | 1 | | 1 | | 1 | | |

### General Method for Preparation of Lipid Formulation:

The following method was used to make lipid composition of the present invention.

For three or four component formulations, all the lipid components were first made stocks in chloroform. Respective volumes of lipids from their stock were mixed in a glass vial (or round bottom flask, if the amount is huge), allowed to mix thoroughly by hand shaking and then chloroform was evaporated by thin flow of nitrogen (or by rotavapor for round bottom flask), and was put under high vacuum for at least 3 hours. To the lipid component mixture was then added minimal ethanol but not more than 5 volume percent with respect to aqueous buffer, when it would be mixed to that suitable buffer solution (pH 7.4) finally. A brief high powered (>80 Hz) bath sonication for 2-3 minutes at room temperature may be necessary to break tiny visible clumps, if any.

### Form of Lipid Composition:

The said CL based formulation as describe above when mixed/complexed with RNA at a fixed mole ratio form lipid/RNA complex or ‘lipoplex’. The final formulation in the lipoplex form.

### Example 3: Characterization of Liposomal Formulations

| | 1 Hour | | | 24 Hour | | | 48 Hour | | | 72 Hour | | |
|---|---|---|---|---|---|---|---|---|---|---|---|---|
| Formu- | SIZE | CHARGE | PDI | SIZE | CHARGE | PDI | SIZE | CHARGE | PDI | SIZE | CHARGE | PDI |
| lations | (nm) | (mV) | (%) | (nm) | (mV) | (%) | (nm) | (mV) | (%) | (nm) | (mV) | (%) |
| CLF 1 | 151 | 55.2 | 19 | 144 | 51.5 | 21 | 141 | 55.2 | 19.8 | 142 | 40 | 17 |
| CLF 2 | 136 | 37 | 16.3 | 138 | 49.5 | 18 | 130 | 42.3 | 21 | 138 | 41.1 | 22.9 |
| CLF 3 | 163 | 60.2 | 28 | 124 | 60.7 | 30 | 117 | 59.9 | 29 | 118 | 55.6 | 26 |
| CLF 4 | 110 | 55.6 | 20 | 110 | 59.9 | 16.9 | 111 | 55 | 21.7 | 108 | 58.7 | 16 |
| CLF 5 | 124 | 57.9 | 22 | 120 | 59.2 | 24 | 120 | 62.8 | 23.6 | 129 | 58.4 | 22 |
| CLF 6 | 86 | 56.1 | 29 | 88 | 64.7 | 25.3 | 83 | 59 | 25.3 | 83 | 63.4 | 26 |
| CLF 7 | 135 | 48 | 35 | 104 | 65.2 | 33.4 | 108 | 44.3 | 25.6 | 97 | 59.7 | 23 |
| CLF 8 | 143 | 60.7 | 7 | 149 | 46.2 | 1.7 | 150 | 58.1 | 8.9 | 147 | 58.1 | 16 |
| CLF 9 | 129 | 54.6 | 15 | 124 | 63 | 18.9 | 130 | 59.6 | 21.3 | 132 | 59.2 | 18 |
| CLF 10 | 118 | 51.5 | 20 | 127 | 60.8 | 19 | 127 | 58 | 19 | 127 | 51.9 | 24 |
| CLF 11 | 112 | 61.7 | 21.4 | 108 | 60.6 | 23 | 111 | 57.1 | 20 | 114 | 53.4 | 18 |
| CLF 12 | 137 | 55.2 | 9.3 | 143 | 56.1 | 7.5 | 143 | 59 | 19 | 147 | 53.2 | 11 |
| CLF 13 | 103 | 56.9 | 23.8 | 91 | 60.8 | 24 | 91 | 59.5 | 24 | 94 | 57.4 | 26 |
| CLF 14 | 134 | 51.6 | 14.2 | 145 | 61.4 | 15.6 | 155 | 62.2 | 11 | 150 | 53.7 | 14 |
| CLF 15 | 196 | 65 | 12.5 | 183 | 61.8 | 15 | 180 | 61.9 | 13 | 179 | 52.9 | 6 |
| CLF 16 | 143 | 37.7 | 12.7 | 146 | 65.9 | 15.8 | 146 | 68.4 | 17 | 143 | 55.1 | 15 |
| CLF 17 | 225 | 60.3 | 18.1 | 225 | 59.9 | 20 | 212 | 57.9 | 21 | 215 | 55.1 | 20 |
| CLF 18 | 184 | 59.2 | 16 | 179 | 60.3 | 12 | 181 | 59.9 | 10.8 | 183 | 54.1 | 16 |

### Example 4: Nano-Luciferase Sequence

| Element | Sequence |
|---|---|
| T7 promoter | TAATACGACTCACTATAGGG (nucleotides 1-20 of |
| | SEQ ID NO: 1) |
| Globin 5 UTR | ACTCTTCTGGTCCCCACAGACTCAGAGAGAACC |
| | CACC (nucleotides 21-57 of SEQ ID NO: 1) |
| MCS | CTCGAGCTCAAGCTTCGAATTCGGTACCG |
| | (nucleotides 58-86 of SEQ ID NO: 1) |
| N-ORF | ATGGTCTTCACACTCGAAGATTTCGTTGGGGAC |
| | TGGCGACAGACAGCCGGCTACAACCTGGACCAA |
| | GTCCTTGAACAGGGAGGTGTGTCCAGTTTGTTTC |
| | AGAATCTCGGGGTGTCCGTAACTCCGATCCAAA |
| | GGATTGTCCTGAGCGGTGAAAATGGGCTGAAGA |
| | TCGACATCCATGTCATCATCCCGTATGAAGGTCT |
| | GAGCGGCGACCAAATGGGCCAGATCGAAAAAA |
| | TTTTTAAGGTGGTGTACCCTGTGGATGATCATCA |
| | CTTTAAGGTGATCCTGCACTATGGCACACTGGT |
| | AATCGACGGGGTTACGCCGAACATGATCGACTA |
| | TTTCGGACGGCCGTATGAAGGCATCGCCGTGTT |
| | CGACGGCAAAAAGATCACTGTAACAGGGACCCT |
| | GTGGAACGGCAACAAAATTATCGACGAGCGCCT |
| | GATCAACCCCGACGGCTCCCTGCTGTTCCGAGT |
| | AACCATCAACGGAGTGACCGGCTGGCGGCTGTG |
| | CGAACGCATTCTGGCGTAA (nucleotides 92-607 of |
| | SEQ ID NO: 1) |
| Globin 3 UTR | GCTGGAGCCTCGGTGGCCATGCTTCTTGCCCCTT |
| | GGGCCTCCCCCCAGCCCCTCCTCCCCTTCCTGCA |
| | CCCGTACCCCCGTGGTCTTTGAATAAAGTCTGA |
| | GTGGGCGGC (nucleotides 616-725 of SEQ ID NO: 1) |
| poly AAAAA | AAAAAAAAAAAAAAAAAAAAAAAAAAAAAAA |
| | A (nucleotides 726-757 of SEQ ID NO: 1) |
| Full Sequence | TAATACGACTCACTATAGGGACTCTTCTGGTCCC |
| | CACAGACTCAGAGAGAACCCACCCTCGAGCTCA |
| | AGCTTCGAATTCGGTACCGCCACCATGGTCTTC |
| | ACACTCGAAGATTTCGTTGGGGACTGGCGACAG |
| | ACAGCCGGCTACAACCTGGACCAAGTCCTTGAA |
| | CAGGGAGGTGTGTCCAGTTTGTTTCAGAATCTC |
| | GGGGTGTCCGTAACTCCGATCCAAAGGATTGTC |
| | CTGAGCGGTGAAAATGGGCTGAAGATCGACATC |
| | CATGTCATCATCCCGTATGAAGGTCTGAGCGGC |
| | GACCAAATGGGCCAGATCGAAAAAATTTTTAAG |
| | GTGGTGTACCCTGTGGATGATCATCACTTTAAG |
| | GTGATCCTGCACTATGGCACACTGGTAATCGAC |
| | GGGGTTACGCCGAACATGATCGACTATTTCGGA |
| | CGGCCGTATGAAGGCATCGCCGTGTTCGACGGC |
| | AAAAAGATCACTGTAACAGGGACCCTGTGGAAC |
| | GGCAACAAAATTATCGACGAGCGCCTGATCAAC |
| | CCCGACGGCTCCCTGCTGTTCCGAGTAACCATC |
| | AACGGAGTGACCGGCTGGCGGCTGTGCGAACGC |
| | ATTCTGGCGTAAGCGGCCGCGCTGGAGCCTCGG |
| | TGGCCATGCTTCTTGCCCCTTGGGCCTCCCCCCA |
| | GCCCCTCCTCCCCTTCCTGCACCCGTACCCCCGT |
| | GGTCTTTGAATAAAGTCTGAGTGGGCGGCAAAA |
| | AAAAAAAAAAAAAAAAAAAAAAAAAAAA (SEQ |
| | ID NO: 1) |

### Example 5: Lipid/Nucleic Acid Complexation at Different Charge Ratios

Gel retardation assay: To check the ability of respective liposome formulations to condense a 5 kb pDNA (considered to be mimicking the size of similar size of RNA of 4-4.5 kb size) while varying the lipid to DNA ratio 1:1, 2:1, 4:1. The better the complexation lesser is the free DNA moving under gel-electrophoretic condition.

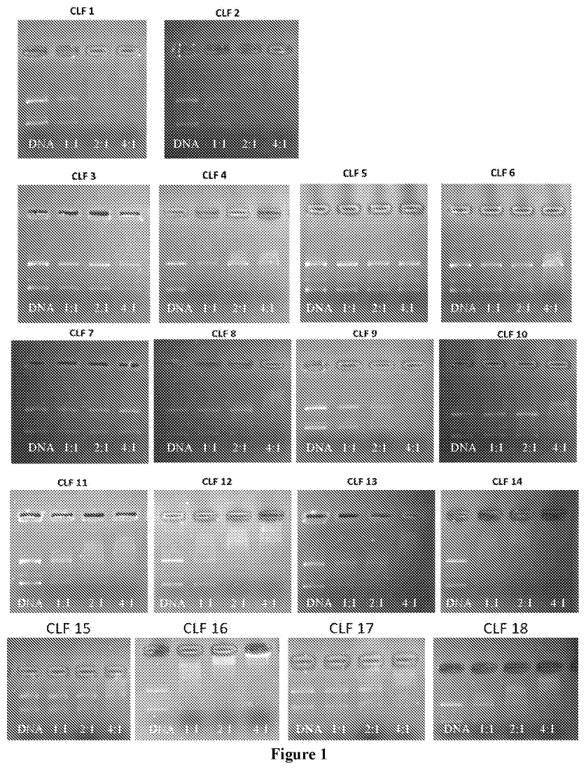

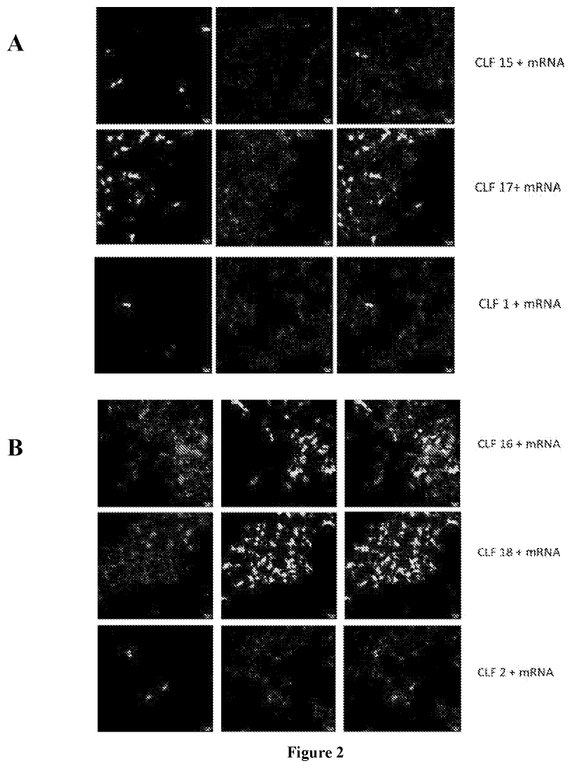

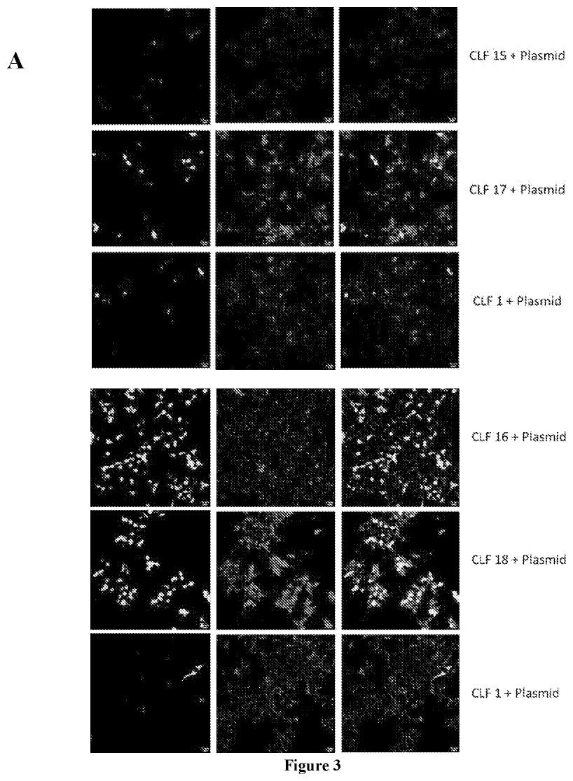

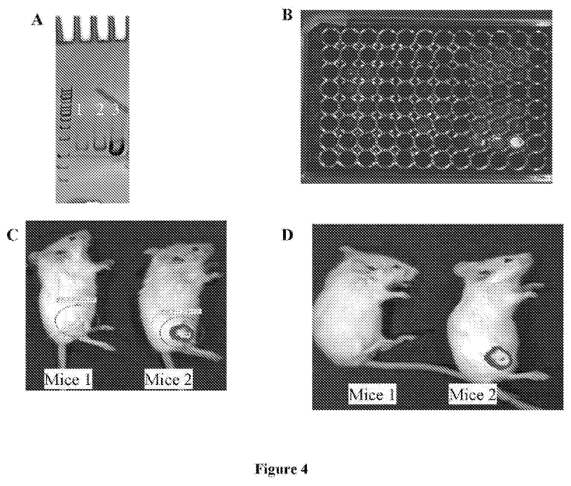

FIG. 1 discloses Gel retardation assay to show level of lipid/nucleic acid (pDNA) complexation with varied charge (N/P) ratio. The more is the complexation between lipid and pDNA, less is the amount of free DNA moving in the gel. The data shows that at more the charge ratios (+/−) of cationic lipid to DNA from 1:1 to 4:1, less is the DNA is moving in the gel. This observation is true in general but the level of free DNA moving in the gel with increased charge ratio (+/−) varies from formulation to formulation, even if the cationic lipid remains same.

Primarily liposomal formulations CLF1, CLF2, CLF15, CLF16, CLF17 and CLF18 has been chosen based on the efficiency of DNA complexation to test for mRNA and pDNA transfection in HEK293 cells.

CLF11, CLF12, CLF13 and CLF14 were also considered to test them in later phase.

### Example 6: MRNA Transfection Assay

Ratio: 4:1 (Charge Ratio or N/P Ratio), mRNA: 50 ng, Cell Line: HEK293 Cells

In this transfection assay, GFP-mRNA is used.

FIG. 2 shows m-RNA transfection using D0 and D1 based lipid formulations with D0 lipid (FIG. 2A) and with D1 lipid (FIG. 2B). It was observed that D1:chol:DOPE (CLF18) is the best formulation so far optimized for RNA transfection in vitro.

CLF2 is CLF18 with DSPE-PEG. It is lesser mRNA transfecting than CLF18 in vitro. Others also showed PEG-lipid indeed reduces the transfection ability of a formulation in vitro. However, previous data by others showed that PEG-lipid is useful for in vivo transfection. So, all these formulations were tested to check their in vivo activity.

Before mRNA transfection, the DNA transfection ability of each of these formulations were also tested.

### Plasmid Transfection Assay: Ratio: 4:1, Plasmid: 50 ng

FIG. 3 shows pDNA DNA transfection ability of D0 (CLF15, CLF17, CLF1) and D1 (CLF16, CLF18, CLF2), with D0 lipid (FIG. 3A) and with D1 lipid (FIG. 3B). The DNA transfection trend is similar to m-RNA transfection, but CLF16 is better than CLF18 in pDNA transfection (unlike mRNA transfection where CLF18 was much better than other formulations especially CLF16).

### Example 7: Construct Development for In Vivo Studies and Optimization

The current study focuses on creating a model platform for mRNA vaccines by designing, synthesis and delivery of various mRNA vaccines with high specificity and superior selectivity with negligible or no allergic reactions.

To standardize the mRNA platform technology, present inventors have designed luciferase construct to make mRNA against luciferase protein.

### Methodology:

Generation of luciferase mRNA construct: To generate luciferase mRNA, CDH-CMV-Nluc-P2A-copGFP-T2A-Puro plasmid were used. The plasmid was digested with EcoR-II and HindIII enzymes to isolate the luciferase gene. Following this the luciferase gene were subcloned into pUC-19 vector (The vector which contains the sequence of T7 promoter, 5′ UTR, multiple cloning sequence, 3′ UTR and 30 base pairs of poly(A)).

Synthesis of mRNA: Nano-Luciferase mRNA (N-Luc mRNA) was generated by transcription in vitro kit (HiScribe™ T7 High Yield RNA Synthesis) with blend of ARCA: 30-O-Me-m7G (50) ppp (50) G (catalog no. [cat. #] N-1081: Trilink Biotechnologies); and NImethylpseudouridine50-triphosphate (cat. #N-1081: Trilink Biotechnologies). The mRNA was purified with the Monarch RNA Cleanup Kit (500 μg), NEB according to the manufacturer's instructions and treated with Antarctic Phosphatase (cat. #M0289L; NEB). It was then repurified with the Monarch® RNA Cleanup Kit. The mRNA was quantified using a NanoDrop spectrometer (Thermo Scientific), precipitated with ethanol and ammonium acetate, and resuspended in 10 mM Tris-HCl and 1 mM EDTA.

Standardization of mRNA Synthesis Using N-Luciferase with Pseudouridine:
N-luc mRNA was synthesized using HiScribe kit (HiScribe™ T7 ARCA mRNA Kit-E2060S) with ARCA and pseudouridine.
N-luc mRNA was purified with NEB kit (Monarch® RNA Cleanup Kit (500 μg)—T2050L) as per the manufacturer's instructions and loaded on to the denaturated formalin 1% agarose gel (A) and transfected into HEK-293T cells and images were captured (B) by adding the Nano-glow substrate (Nano-Glo®) In Vivo Substrate, FFz).

FIG. 4A shows mRNA synthesis, wherein
1. N-Luc-mRNA (with ARCA)—0.4 μg+POLY (A)
2. N-Luc-mRNA (with ARCA)—1 μg+POLY (A)
3. N-Luc-mRNA (with ARCA)—without POLY (A)

FIG. 4B shows Expression of N-Luc protein in mRNA transfected cells (N luc mRNA 1 sec)

Further, N-Luc mRNA was encapsulated with CLF18 and injected into BALBc mice (mice 2) mouse rectus femoris muscle. Only vehicle (CLF 18 formulation) was injected into BALBc mice (mice 1) mouse rectus femoris muscle. After 24 hours of mRNA administration, Nanoglow substrate (50 μL of 1× solution) was injected via i.p route and waited for 10 minutes. Then animals were imaged using IVIS spectrum (PerkinElmer). FIG. 4C shows Expression in in vivo (Day 1: N luc mRNA in Balb C mice)

Luciferase expression in right side mice 2 was observed, and there were no signal from mice 1. It indicated that N-Luc expression was observed in in vivo system. It also indicates that Luc protein was being made upon mRNA delivery into mice. Further the animals were imaged again on day 3 and observed the N-luc expression. FIG. 4D shows Expression in in vivo (Day 3: N luc mRNA in Balb C mice).

### Example 8: Lipid/m-RNA Lipoplex Standardization In Vivo

2 μg of N-luc mRNA was mixed with 7 formulations (CLF1, CLF2, CLF15, CLF16, CLF17, CLF18 and M2) of which CLF1, CLF2, CLF15, CLF16, CLF17, CLF18 at 0.1 mM in 1:3 ratio (M2− is 1:2) and incubated. (CLF1 & CLF2 contain PEG lipid: CLF15, CLF16, CLF17 & CLF18 do not contain PEG: M2 is standard formulation for m-RNA delivery).

After incubation N-Luc mRNA-Lipid complexes were administered into mice right and left thigh by intra-muscular injection into 9 mice (only one side for water).

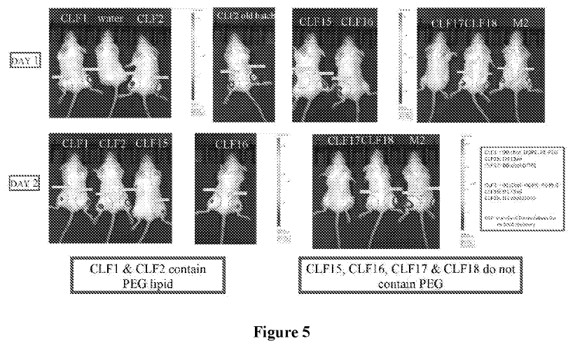

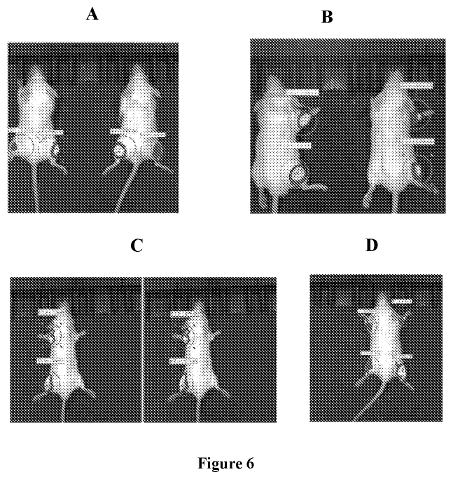

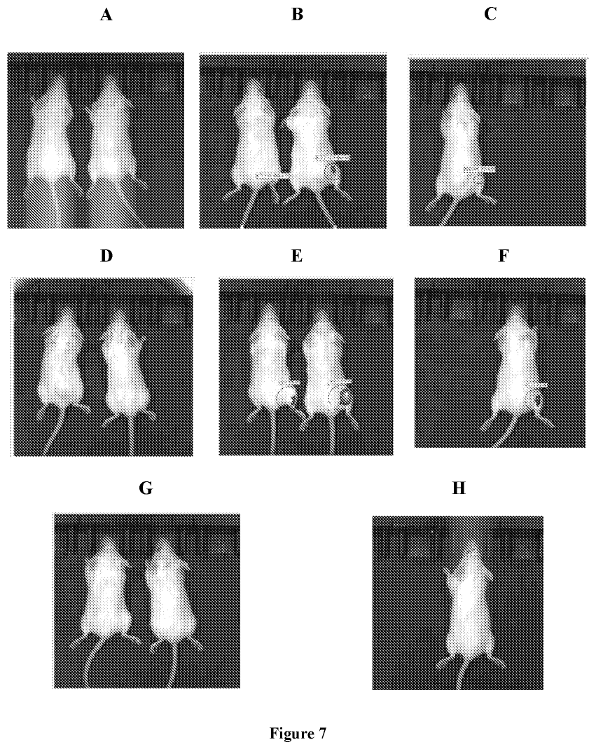

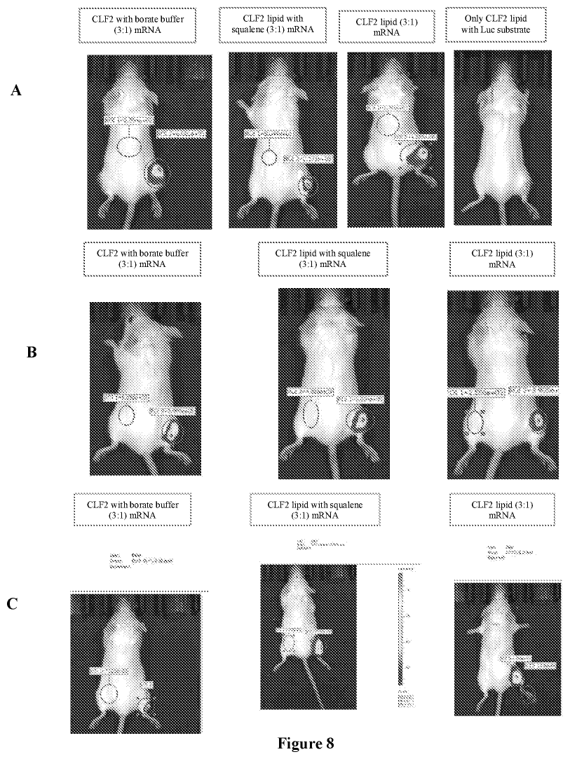

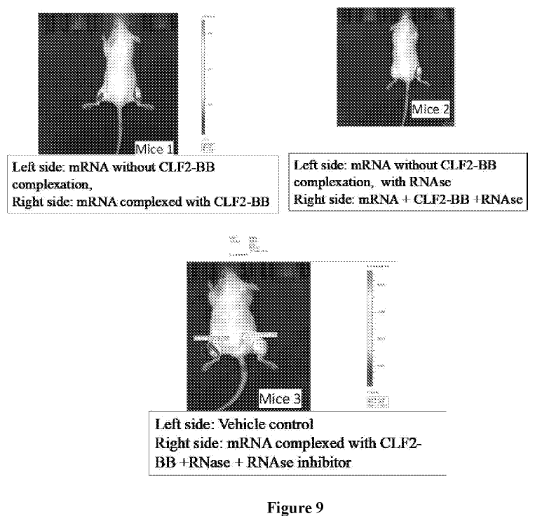

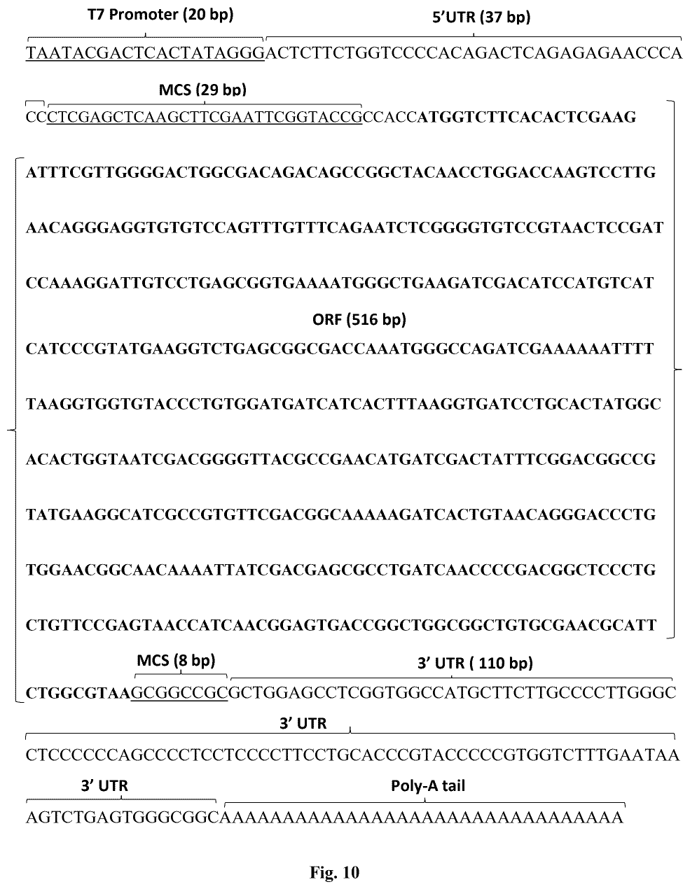

After 24 hours of administration, animals were imaged to check the luciferase expression. FIG. 5 shows Expression of N-Luc protein in vivo on day 1 and 2 post injection.

N-Luc expression in 7 mice were observed, however best signal was observed with CLF2, CLF18 and M2 formulations.

CLF2 and CLF18 lipids showed higher efficacy in terms of luciferase expression in animal models among other lipids tested so far.

### Example 9: Stability Study for mRNA-Lipid Complex

Stability assay was designed and conducted as per below conditions:

N-luc mRNA (2 μg/32 μL) was complexed with CLF18 (18 μL) or CLF2 (18 μL) formulation and these complexes mixed thoroughly with pipetting and kept for shaking at room temperature in dark condition for 1 hour. These complexes were made in duplicates as 5 sets. After one hour of incubation on shaker, mRNA-lipid complexes were incubated at room temperature for specified time period. After specified incubation, mRNA lipid complexes were administered into mouse rectus femoris muscle.
1^{st} set for 30 min stability at room temperature: After complexation, complex was stored at room temperature for 30 min and administered into animals.
2^{nd} set for 24 hours stability at room temperature: After complexation, complex was stored at room temperature for 24 hours and administered into animals.
3^{rd} set for 72 hours stability at room temperature: After complexation, complex was stored at room temperature for 72 hours and administered into animals.
4^{th} set for 10 days stability at room temperature: After complexation, complex was stored at room temperature for 10 days and administered into animals.
5^{th} set for 30 days stability at room temperature: After complexation, complex was stored at room temperature for 30 days and administered into animals.

After specified incubations, complexes were injected into mice and imaged after 24 hours of the administrations to monitor the luciferase expression.

FIG. 6 shows in vivo study to depict the stability of N-luc mRNA while remain in complex with lipids CLF2 and CLF18 with respect to time at room temperature.
A) 30 min stability: left mice (none) CLF18; right mice left ear cut CLF 2
B) 24 hours stability: left mice (none) CLF18; right mice left ear cut CLF 2
C) 3^{rd} day stability: left mice (none) CLF18; right mice left ear cut CLF 2
D) 30^{th} day stability: Left side injected with CLF 18 formulation and right side injected with CLF-2 formulation

It was observed that mRNA complexed with CLF2 or CLF18 formulations were able to form luciferase protein in in vivo conditions. These results revealed that, these complexes are indeed stable and at room temperature till 30 days.

Therefore characterization on stability of lipoplex, N-Luc mRNA complexed with

CLF2 or CLF18 have showed very high stability of m-RNA, i.e., m-RNA remains in vivo transfection competent till 30 days at room temperature.

### Example 10: To Study the Extent of Residence for Lipid Over a Period of Time Following IM Injection

Biodistribution of lipid formulation: To investigate the distribution of lipid molecules in vivo, biodistribution assay with BALB/c animals were performed. Lipid formulation (18 μl) containing (DiR dye) was administered into each animal and imaging was performed after 24 hours, 5 days and 10 days.

FIG. 7 shows In vivo study to depict the extent of residence for lipid over a period of time following IM injection
A) Day 1: Lipid without Dir Dye
B) Day 1: CLF2
C) Day 1: CLF18
D) Day 5: Lipid without Dir Dye
E) Day 5: CLF2
F) Day 5: CLF18
G) Day 15: CLF2
H) Day 15: CLF18

High signal of DiR was observed only in injection site on day 1 with both lipid formulations (B and C) and mild signal were observed on day 5 with CLF2 (E) but not CLF18 (F).

No signal was observed in DiR-free lipid on day 1 or day 5 (A and D). Further imaging on day 15, revealed that, both DiR signal was not obtained in either CLF2 or CLF18 lipid formulations (G and H).

It was observed that the locally injected lipids CLF2 and CLF18 (with DiR dye) could be traced clearly until 5 days post-injection. No lipids signals could be detected on 15th day.

Further excretion analysis experiments are needed to be conducted to confirm the elimination of the lipids.

### Example 11: Stability of Luciferase Expression in In Vivo

After complexation, mRNA lipid complex was injected into animals and images were captured for 24 hours, 7 days and 30 days to check the expression of luciferase protein.

FIG. 8 shows Expression of luciferase protein
A) Imaged after 24 hours of injection
B) Imaged after 7 days of injection
C) Imaged after 30 days of injection

It was observed that the Luciferase expression was stable for one month in in vivo.

mRNA complexed with CLF-2 BB or squalene or only CLF2 showed N-luc expression upto 30 days. However, detection of N-luc expression with CLF2 BB lipid complexed mRNA is faster than squalene or only CLF2 complexed lipids.

### Example 12: Stability of CLF2-Borate Buffer (BB) Complexed mRNA

To investigate the effect of RNAses on mRNA alone and mRNA complexed with CLF-2 borate buffer, stability experiment was performed using N-luc mRNA.

Following conditions were employed in the experiment:
Condition 1: N-Luc mRNA (500 ng) alone incubated for 30 minutes and injected into left side gastronomes muscle of mice 1
Condition 2: N-Luc mRNA (500 ng) complexed with CLF2 BB lipid and incubated for 30 minutes and injected into right side gastronomes muscle of mice 1
Condition 3: N-Luc mRNA (500 ng) alone incubated for 30 minutes, then 25 ng of RNAse was added and further incubated at 37 degrees for another 30 minutes. Then mRNA was injected into left side gastronomes muscle of mice 2.
Condition 4: N-Luc mRNA (500 ng) complexed with CLF2 BB lipid, then 25 ng of RNAse was added and further incubated at 37 degrees for another 30 minutes. Then mRNA was injected into right side gastronomes muscle of mice 2
Condition 5: N-Luc mRNA (500 ng) complexed with CLF2 BB lipid, then 25 ng of RNAse was added and further incubated at 37 degrees for another 30 minutes. Thereafter RNASe inhibitor was added and incubated for another 30 minutes. Then mRNA was injected into left side gastronomes muscle of mice 3
Condition 6: Negative control, only water was injected into right side gastronomes muscle of mice 3.

FIG. 9 shows Effect of RNAses on mRNA alone and mRNA complexed with CLF-2 borate buffer.

It was observed that CLF2-borate buffer (BB) complexed mRNA showed stability in presence of RNAse as well.

### Example 13: Vaccine Formulation

### Method for Preparation of Vaccine:

A method for preparation of vaccine formulation, comprising the steps of:
a) preparation of RNA construct by in vitro transcription process:
b) complexing the CL based formulation as describe above with RNA at a fixed mole ratio to make lipid/RNA complex or ‘lipoplex’:
c) obtaining the lipoplex as the vaccine solution and preserving it at room temperature or at 4° C.

In the vaccine formulation the mRNA is derived from a virus which may be selected from the group comprising Coronaviridae, Retroviridae, Reoviridae, Togaviridae, Filoviridae, Flavirideae, Papillomaviridae, Plasmodiidae, Hepadnaviridae, Picornaviridae, Caliciviridae, Orthomyxoviridae, Adenoviridae, Rhabdoviridae, Paramyxoviridae, Orthomyxoviridae, Nodaviridae etc.

### ADVANTAGES

1. Even at very low charge ratio significant luciferase mRNA expression is observed in vivo (in BALB/c mice)
2. Good lipoplex Stability even at Room Temperature for a month
3. m-RNA-luc expression is obtained even in 100 ng and less the formulation is optimized for lower mole ratio (N/P) as low as 1:1 and above
4. Further it has been confirmed the lipids were traced until 5 days post-injection and no lipids signals could be detected on 15th day. Thus lipid-mediated toxicity was minimized by its short time body exposure by elimination of the lipids.

1. A cationic lipid (CL) based composition for nucleic acid (NA) delivery, wherein the CL-based composition comprises:
(a) Cationic lipid: N-oleyl, N-octadecyl, N, N dihydroxyethyl ammonium chloride [lipid D1] or its salt, solvate, derivatives, or combination thereof;
(b) combination of Neutral/Zwitter-ionic co-lipids which comprises two or more lipids selected from:
(b1) steroid lipid which may be sterol and is selected from Cholesterol, ergosterol, stigmasterol, sitosterol, campesterol, stigmastanol like phytosterols and other steroids such as dexamethasone, prednisolone, triamcinolone and/or mixture thereof;
(b2) phospholipid selected from Dioleoyl phosphatidylethanolamine [DOPE], 1,2-Dioleoyl-sn-Glycero-3-Phosphocholine [DOPC], 1,2-Distearoyl-sn-glycero-3-phosphoethanolamine [DSPE], 1,2-Distearoyl-sn-glycero-3-phosphocholine [DSPC], 1,2-Dimyristoyl-sn-glycero-3-phosphoethanolamine [DMPE], 1,2-Dimyristoyl-sn-glycero-3-phosphocholine [DMPC], 1,2-Dipalmitoyl-sn-glycero-3-phosphoethanolamine [DPPE], 1,2-Dipalmitoyl-sn-glycero-3-phosphocholone [DPPC] or mixture thereof, preferably DOPE and/or DOPC; and
(b3) a phospholipid-polymer conjugate selected from conjugates prepared by conjugation of a suitable phospholipid selected from Dioleoyl phosphatidylethanolamine [DOPE], 1,2-Distearoyl-sn-glycero-3-phosphoethanolamin [DSPE], 1,2-Dimyristoyl-sn-glycero-3-phosphoethanolamine [DMPE], 1,2-Dipalmitoyl-sn-glycero-3-phosphoethanolamin [DPPE], with a suitable polymer which is selected from polyethylene-glycol [PEG] of 2000, 5000, 8000 Average molecular weight, and
(c) optionally, one or more pharmaceutically acceptable excipient.

2. The cationic lipid (CL) based composition for nucleic acid (NA) delivery as claimed in claim 1, wherein the steroid lipid (b1) is cholesterol.

3. The cationic lipid (CL) based composition for nucleic acid (NA) delivery as claimed in claim 1, wherein the phospholipid (b2) is Dioleoyl phosphatidylethanolamine [DOPE].

4. The cationic lipid (CL) based composition for nucleic acid (NA) delivery as claimed in claim 1, wherein the Phospholipid-Polymer conjugate (b3) is 1,2-distearoyl-sn-glycero-3-phosphoethanolamine-N-[amino(polyethylene glycol)-2000 [DSPE-PEG].

5. The cationic lipid (CL) based composition for nucleic acid (NA) delivery as claimed in claim 1, wherein the composition comprises:
(a) cationic lipid N-oleyl, N-octadecyl, N, N dihydroxyethyl ammonium chloride [lipid-D1];
(b) neutral/zwitter-ionic co-lipids selected from (b1) Cholesterol, (b2) Dioleoylphosphatidylethanolamine [DOPE], with or without Phospholipid-Polymer conjugate (b3) 1,2-distearoyl-sn-glycero-3-phosphoethanolamine-N-[amino(polyethylene glycol)-2000 [DSPE-PEG]; and
(c) optionally, one or more pharmaceutically acceptable excipient.

6. The cationic lipid (CL) based composition for nucleic acid (NA) delivery as claimed in claim 5, wherein the mole ratio of N-oleyl, N-octadecyl, N, N dihydroxyethyl ammonium chloride [Lipid-D1], Cholesterol, 1,2-Dioleoyl-sn-glycero-3-phosphoethanolamine [DOPE] in the composition is 1:1:1.

7. The cationic lipid (CL) based composition for nucleic acid (NA) delivery as claimed in claim 5, wherein the mole ratio of N-oleyl, N-octadecyl, N, N dihydroxyethyl ammonium chloride [Lipid-D1], Cholesterol, 1,2-Dioleoyl-sn-glycero-3-phosphoethanolamine [DOPE], and 1,2-distearoyl-sn-glycero-3-phosphoethanolamine-N-[amino(polyethylene glycol)-2000 [DSPE-PEG] in the composition is 1:1:1:0.03.

8. The cationic lipid (CL) based composition for nucleic acid (NA) delivery as claimed in claim 1, wherein the Nucleic Acid (NA) is a biologically active agent which is delivered to a subject in need thereof using the said CL composition, wherein the Nucleic Acid (NA) is selected from ribonucleic acid (RNA), messenger RNA (mRNA), Deoxyribonucleic acid (DNA), plasmid DNA (pDNA), fragment of RNA, mRNA, DNA, pDNA, miRNA or any chimeric or fusion thereof.

9. The cationic lipid (CL) based composition for nucleic acid (NA) delivery as claimed in claim 8, wherein the Nucleic Acid (NA) is derived from a living bio-organism and non-living bio-organism.

10. The cationic lipid (CL) based composition for nucleic acid (NA) delivery as claimed in claim 9, wherein the Nucleic Acid (NA) is derived from a virus selected from Coronaviridae, Retroviridae, Reoviridae, Togaviridae, Filoviridae, Flavirideae, Papillomaviridae, Plasmodiidae, Hepadnaviridae, Picornaviridae, Caliciviridae, Orthomyxoviridae, Adenoviridae, Rhabdoviridae, Paramyxoviridae, Orthomyxoviridae and Nodaviridae.

11. The cationic lipid (CL) based composition for nucleic acid (NA) delivery as claimed in claim 8, wherein the biologically active agent is a DNA, pDNA, RNA, mRNA derived from the virus selected from Coronaviridae, Retroviridae, Reoviridae, Togaviridae, Filoviridae, Flavirideae, Papillomaviridae, Plasmodiidae, Hepadnaviridae, Picornaviridae, Caliciviridae, Orthomyxoviridae, Adenoviridae, Rhabdoviridae, Paramyxoviridae, Orthomyxoviridae and Nodaviridae.

12. The cationic lipid (CL) based composition as claimed in claim 8, wherein the composition for biologically active Nucleic Acid (NA) delivery is formulated in a suitable lipid formulation preparation as a delivery system with or without one or more pharmaceutically acceptable excipient, wherein the suitable lipid formulation may be selected from:
Nanoparticle formulation,
Lipid/Liposome/Liposomal formulation,
Lipoplex formulation,
Lipid/polymer emulsion formulation.

13. The cationic lipid (CL) based composition as claimed in claim 12, wherein the composition is formulated as a cationic lipid (CL) based formulation formulated along with the biologically active Nucleic Acid (NA) to provide a pharmaceutical formulation with or without one or more pharmaceutically acceptable excipient, to administer in a subject in need thereof, wherein the pharmaceutical formulation comprises:
a Lipid-Nanoparticle (LNP) formulation or a Liposomal formulation—encapsulating the biologically active agent, or
a Lipoplex formulation—formed by complexion of Liposomal preparation with the biologically active agent.

14. The cationic lipid (CL) based composition as claimed in claim 13, wherein the pharmaceutically acceptable excipients are selected buffers, adjuvants, diluents, lubricants, binders, stabilizers, preservatives, disintegrants, absorbents, colorants surfactants, flavors, sweeteners. residuals, immune boosting co-excipients or combination thereof.

15. The cationic lipid (CL) based composition as claimed in claim 13, wherein the pharmaceutical formulation is a vaccine formulation for delivery of biologically active agent which comprises:
a complex of Lipid composition+DNA/pDNA (DNA/pDNA-Lipoplex), or
a complex of Lipid composition+RNA/mRNA complex (RNA/mRNA-Lipoplex).

16. The cationic lipid (CL) based composition as claimed in claim 15, wherein the formulation performs in vivo transfection at a N/P charge ratio (Lipid: Nucleic acid) of 1:1 to 4:1.

17. The cationic lipid (CL) based composition as claimed in claim 16, wherein the N/P charge ratio is 1:1 or 2:1 or 3:1 or 4:1.

18. The cationic lipid (CL) based composition as claimed in claim 15, wherein the formulation is a lipid/mRNA complex (lipoplex) which is stable at room temperature for at least 30 days.

19. (canceled)

20. (canceled)

21. A vaccine formulation comprising nucleic acid as vaccine antigen for the prophylaxis and/or treatment of virus-mediated disease in a human subject, wherein the vaccine comprises:
a Nucleic acid (NA) based biologically active vaccine antigen selected from DNA, pDNA, RNA, mRNA, fragment, or combination thereof derived from virus;
a cationic-based (CL)—lipid composition which comprises
a. Cationic lipid: N-oleyl, N-octadecyl, N, N dihydroxyethyl ammonium chloride [lipid D1] or its salt, solvate, derivatives, or combination thereof; and
b. combination of Neutral/Zwitter-ionic co-lipids which comprises two or more lipids selected from:
(b1) steroid lipid which may be sterol and is selected from Cholesterol, ergosterol, stigmasterol, sitosterol, campesterol, stigmastanol like phytosterols and other steroids such as dexamethasone, prednisolone, triamcinolone and/or mixture thereof;
(b2) phospholipid which may be selected from Dioleoyl phosphatidylethanolamine [DOPE], 1,2-Dioleoyl-sn-Glycero-3-Phosphocholine [DOPC], 1,2-Distearoyl-sn-glycero-3-phosphoethanolamine [DSPE], 1,2-Distearoyl-sn-glycero-3-phosphocholine [DSPC], 1,2-Dimyristoyl-sn-glycero-3-phosphoethanolamine [DMPE], 1,2-Dimyristoyl-sn-glycero-3-phosphocholine [DMPC], 1,2-Dipalmitoyl-sn-glycero-3-phosphoethanolamine [DPPE], 1,2-Dipalmitoyl-sn-glycero-3-phosphocholone [DPPC] or mixture thereof, preferably DOPE and/or DOPC; and
(b3) a phospholipid-polymer conjugate which may be selected from conjugates prepared by conjugation of a suitable phospholipid selected from Dioleoyl phosphatidylethanolamine [DOPE], 1,2-Distearoyl-sn-glycero-3-phosphoethanolamin [DSPE], 1,2-Dimyristoyl-sn-glycero-3-phosphoethanolamine [DMPE], 1,2-Dipalmitoyl-sn-glycero-3-phosphoethanolamin [DPPE], with a suitable polymer which is selected from polyethylene-glycol [PEG] of 2000, 5000, 8000 Average molecular weight, and
c. optionally, one or more pharmaceutically acceptable excipient.

22. The vaccine formulation as claimed in claim 21, wherein the vaccine antigen DNA, pDNA, RNA, mRNA, fragment, or combination thereof are derived from virus selected from the group comprising Coronaviridae, Retroviridae, Reoviridae, Togaviridae, Filoviridae, Flavirideae, Papillomaviridae, Plasmodiidae, Hepadnaviridae, Picornaviridae, Caliciviridae, Orthomyxoviridae, Adenoviridae, Rhabdoviridae, Paramyxoviridae, Orthomyxoviridae or Nodaviridae.

23. The vaccine formulation as claimed in claim 21, wherein the vaccine antigen is mRNA formulated with a suitable lipid formulation for delivery of active antigenic agent which comprises:
(a) cationic lipid N-oleyl, N-octadecyl, N, N dihydroxyethyl ammonium chloride [lipid-D1];
(b) neutral/zwitter-ionic co-lipids selected from (b1) Cholesterol, (b2) Dioleoylphosphatidylethanolamine [DOPE], with or without Phospholipid-Polymer conjugate (b3) 1,2-distearoyl-sn-glycero-3-phosphoethanolamine-N-[amino(polyethylene glycol)-2000 [DSPE-PEG]; and
(c) optionally, one or more pharmaceutically acceptable excipient.

24. The vaccine formulation as claimed in claim 23, wherein the mole ratio of N-oleyl, N-octadecyl, N, N dihydroxyethyl ammonium chloride [Lipid-D1], Cholesterol, 1,2-Dioleoyl-sn-glycero-3-phosphoethanolamine [DOPE] in the composition of the vaccine formulation is 1:1:1.

25. The vaccine formulation as claimed in claim 23, wherein the mole ratio of N-oleyl, N-octadecyl, N, N dihydroxyethyl ammonium chloride [Lipid-D1], Cholesterol, 1,2-Dioleoyl-sn-glycero-3-phosphoethanolamine [DOPE], and 1,2-distearoyl-sn-glycero-3-phosphoethanolamine-N-[amino(polyethylene glycol)-2000 [DSPE-PEG] in the composition of the vaccine formulation is 1:1:1:0.03.

26. The vaccine formulation as claimed in claim 23,
wherein the suitable lipid formulation, may be selected from:
Nanoparticle formulation,
Lipid/Liposome/Liposomal formulation,
Lipoplex formulation,
Lipid/polymer emulsion formulation.

27. The vaccine formulation as claimed in claim 26, wherein the suitable lipid formulation is selected from:
a Lipid-Nanoparticle (LNP) formulation or a Liposomal formulation—encapsulating the mRNA,
a Lipoplex formulation—formed by complexion of Liposomal preparation with mRNA.

28. The vaccine formulation as claimed in claim 21, wherein the vaccine formulation performs in vivo transfection at a N/P charge ratio (Lipid:mRNA) of 1:1 to 4:1.

29. The vaccine formulation as claimed in claim 28, wherein the N/P charge ratio is 1:1 or 2:1 or 3:1 or 4:1.

30. The vaccine formulation as claimed in claim 21, wherein the formulation is stable at room temperature for at least 30 days.

31. The vaccine formulation as claimed in claim 21, wherein the vaccine minimizes lipid-mediated toxicity in a subject administered with vaccine in need thereof, by minimizing the amount of cationic-lipid required in the composition/formulation of the vaccine.

32. The vaccine formulation as claimed in claim 21, may be formulated with or without one or more pharmaceutically acceptable excipient.

33. The vaccine formulation as claimed in claim 32, wherein the pharmaceutically acceptable excipients are selected from buffers, adjuvants, diluents, lubricants, binders, stabilizers, preservatives, disintegrants, absorbents, colorants surfactants, flavors, sweeteners. residuals, immune boosting co-excipients or combination thereof.

34. The vaccine formulation as claimed in claim 21, wherein the formulation is in liquid, powder, lyophilized or suspension form.

35. A method for preparation of a cationic lipid (CL) based composition of claim 1, comprising the steps of:
a) taking CL and other co-lipids and excipients first in chloroform and mixing thoroughly in a vial;
b) drying off chloroform by thin layer of N2 flow, followed by keeping the vial at high vacuum; and
c) dissolving the residual lipid mixture in minimal ethanol followed by rapidly injecting the ethanolic lipid mixture in the suitable buffer solution, typically of pH 7.4, to make the CL based formulation in room temperature.

36. A method for preparation of vaccine formulation of claim 21, comprising the steps of:
a) preparation of RNA construct by in vitro transcription process;
b) complexing the CL based formulation as claimed in claim 1 with RNA at a fixed mole ratio to make lipid/RNA complex or ‘lipoplex’;
c) obtaining the lipoplex as the vaccine solution and preserving it at room temperature or at 4° C.

37. A method of prophylaxis, treatment, and management of virus-mediated disease by administering e vaccine formulation of claim 21 in a subject in the need thereof by intranasal, intraperitoneal, oral, intramuscular, subcutaneous or intradermal routes.

38. Use of cationic lipid (CL) based composition of claim 1 for:
delivery of Nucleic acid;
minimizing toxicity of cationic lipid;
in preparation of pharmaceutical formulation including vaccine formulation;
in prophylaxis, treatment, and management of diseases caused by living and non-living microorganism including bacteria and viruses.


SEQUENCE LISTING (1 sequence)

SEQ ID NO: 1 - DNA, 757 bp, synthetic construct
  source 1..757: mol_type = other DNA
taatacgactcactatagggactcttctggtccccacagactcagagagaacccaccctcgagctcaagcttcgaattcggtaccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagcggccgcgctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaa